(19) United States
(12) Patent Application Publication
(10) Pub. No.: US 2023/0357154 A1
(43) Pub. Date: Nov. 9, 2023

(54) BUTYRYLCHOLINESTERASE COMPOUNDS AND USE IN DISEASES OF THE NERVOUS SYSTEM

(71) Applicant: Treventis Corporation, Bernville, PA (US)

(72) Inventor: Sultan Darvesh, Halifax (CA)

(73) Assignee: Treventis Corporation, Bernville, PA (US)

(21) Appl. No.: 17/592,942

(22) PCT Filed: Aug. 7, 2020
(86) PCT No.: PCT/US2020/045357

§ 371 Date: Feb. 4, 2022

Related U.S. Application Data
(60) Provisional application No. 62/884,442, filed on Aug. 8, 2019.

(51) Int. Cl. C07D 213/79; C12Q 1/46; C07D 221/00; A61K 51/04

(52) U.S. Cl. CPC C07D 213/79; C12Y 301/01008; C12Q 1/46; C07D 221/00; A61K 51/0455

(57) ABSTRACT

In general, among other things, compounds of Formula I are provided:

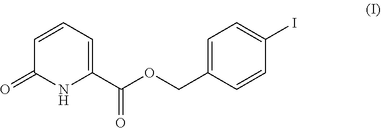 [SMILES: O=C(OCc1ccc(I)cc1)c1cccc(=O)[nH]1]

or a pharmaceutically acceptable salt thereof. Other compounds are also provided. Methods of diagnosis and treatment are also provided.

### BACKGROUND OF THE INVENTION

In Alzheimer's disease (AD) there are three major microscopic features in the brain that are recognized as the hallmarks of the disease, namely neuritic plaques (NP), neurofibrillary tangles (NFT) and amyloid angiopathy (AA). In addition, there is widespread cell loss, particularly of cholinergic neurons in the brain. Loss of cholinergic cells leads to reduction in the levels of the neurotransmitter acetylcholine, its synthesizing enzyme choline acetyltransferase, as well as its deactivating enzyme acetylcholinesterase (AChE, EC number 3.1.1.7). Reduction of cholinergic neurotransmission leads to some of the symptoms of AD.

Butyrylcholinesterase (BuChE) is found to have a brain distribution pattern that is distinct from that of acetylcholinesterase (AChE). Neurons containing BuChE are particularly located in the amygdala, hippocampal formation and the thalamus, structures involved in the normal functions of cognition and behavior that typically become compromised in Alzheimer's disease (AD). In the normal brain BuChE is mainly expressed in white matter, glia and distinct subcortical populations of neurons important for cognition and behavior. See e.g. S. Darvesh, D. L. Grantham, D. A. Hopkins, Distribution of butyrylcholinesterase in the humanamygdala and hippocampal formation, J Comp Neurol. 393 (1998) 374-390; S. Darvesh, D. A. Hopkins, Differential distribution of butyrylcholinesterase and acetylcholinesterase in the human thalamus, J Comp Neurol. 463 (2003a) 25-43; S. Darvesh, G. Geula, D. A. Hopkins, Neurobiology of butyrylcholinesterase, Nature Reviews Neuroscience. 4 (2003b) 131-138. AChE, in contrast, is found in neurons and neuropil throughout the brain. See e.g. M. Mesulam, C. Geula, Chemoarchitectonics of axonal and perikaryal acetylcholinesterase along information processing systems of the human cerebral cortex, Brain. In AD, both cholinesterases associate with Aβ plaques and NFTs. The accumulation of BuChE in AD pathology is especially notable in cortical grey matter, an area that normally has very little BuChE activity.

Although the level of AChE is reduced in AD, the level of the closely related enzyme butyrylcholinesterase (BuChE, EC number 3.1.1.8) is increased in AD brain. BuChE is found in the neuropathological lesions associated with AD, namely, NP, NFT and AA. Importantly, BuChE is found in NP in brains of patients with AD. BuChE is found in a higher number of plaques in brains of elderly individuals with AD relative to those without AD. It has been shown that some BuChE inhibitors not only improve cognition in an animal model but also reduce the production of β-amyloid, which is one of the principal constituents of neuritic plaques.

From a neuropathology perspective, deposition of amyloid and formation of NP is one of the central mechanisms in the evolution of AD. However, amyloid plaques are also found in brains of elderly individuals who do not have dementia (Guillozet et al., 1997). It has been suggested that the amyloid plaques in individuals without dementia are “benign” and they become “malignant”, causing dementia, when they are transformed into plaques containing degenerated neurites. These plaques are called neuritic plaques (NP). The mechanism of transformation from “benign” to “malignant” plaques is as yet unknown. It has been suggested that BuChE may play a major role in this transformation based on the observation that BuChE is found predominately in plaques that contain dystrophic neurites and not in plaques without dystrophic neurites.

Taken together, these observations suggest that in the brains of patients with AD there is a significant alteration of the biochemical properties of BuChE that alters its normal regulatory role in the brain, thus contributing to the pathology of AD. A compound that can modulate BuChE would therefore be useful as a therapeutic or diagnostic for AD. There remains a need for such compounds.

Multiple sclerosis (MS) is a neuroinflammatory and neurodegenerative disease of the central nervous system. MS manifests as a progressive loss of physical and cognitive faculties thought to be a result of widespread demyelination within the brain. Current methods for diagnosis of MS rely upon presentation of clinical symptoms as well as MRI imaging of lesions in the brain. MRI is a sensitive approach for the visualization of MS lesions however, it remains a non-specific methodology and thus additional evidence is required to reach a diagnosis. There remains a need for MS-specific imaging agents in order to provide an early and definitive diagnosis of this disease. Early diagnosis is crucial as several disease modifying therapies have been proven effective for MS Primary brain tumours are the result of aberrant proliferation of brain cells. Tumours of this nature can cause an enormity of clinical symptoms dependent upon location and size within the brain. Several non-specific imaging approaches are used to visualize tumours, such as MRI. However, a method to specifically detect tumours at an early stage has not heretofore been reported.

The first cholinesterase inhibitor (ChEI) was introduced in 1997 and it has been known that certain chemical compounds such as carbamates had anticholinesterase activity even earlier. For example in 1995, it was known that “in cognitive responders, memory enhancement by physostigmine in Alzheimer's disease is correlated directly to the magnitude of plasma cholinesterase inhibition.” Sanjay Asthana MD, Clinical pharmacokinetics of physostigmine in patients with Alzheimer's disease Clinical Pharmacology & Therapeutics (1995) 58, 299-309.

Anticholinesterases such as the cholinergic drugs, donepezil, galantamine and the carbamate, rivastigmine, are now considered by many to be the first line pharmacotherapy for mild to moderate Alzheimer's disease enhance cognitive function and are known to act by enhancing cholinergic function in the brain. Birks J. Cholinesterase inhibitors for Alzheimer's disease, Cochrane Database of Systematic Reviews 2006, Issue 1, Art. No.: CD005593. DOI: 10.1002/14651858.CD005593. These drugs have slightly different pharmacological properties, but are thought to all work by inhibiting the breakdown of acetylcholine by blocking the enzyme acetylcholinesterase.

Alzheimer's patients often exhibit other symptoms including depression, anxiety and sleep disorders, all of which may benefit from treatment with acetylcholinesterase inhibitors, such as the carbamates rivastigmine and physostigmine.

However, the carbamate physostigmine has not been well tolerated by patients and therefore cannot be used clinically for treatment of Alzheimer's disease. One of the reasons for this is that physostigmine is an extremely powerful inhibitor of cholinesterases in that it deactivates both AChE and BuChE extremely fast, raising acetylcholine levels rapidly. For this reason, patients treated with physostigmine experience significant intolerable side effects.

Rivastigmine, on the other hand, deactivates cholinesterases slower relative to physostigmine, but still not slow enough to avoid side effects. The rivastigmine patch was developed to affect a slower release of the rivastigmine to overcome this problem and has been shown to have lessened the side effects because of slower rate of release of the drug and hence deactivation of cholinesterase.

Research to find carbamates having the desired anticholinesterase activity but with less of the undesirable characteristics of carbamates, such as high toxicity, narrow therapeutic window and short duration of action has continued. See e.g. Qian-Sheng Yu, Carbamate analogues of (−)-physostigmine: In vitro inhibition of acetyl-and butyrylcholinesterase, Feb Letter, Volume 234, Issue 1, 4 Jul. 1988, Pages 127-130; Maruyama W, Anti-apoptotic action of anti-Alzheimer drug, TV3326 [(N-propargyl)-(3R)-aminoindan-5-yl]-ethyl methyl carbamate, a novel cholinesterase-monoamine oxidase inhibitor. Neurosci Lett. 2003 May 8; 341(3):233-6.

According to U.S. Pat. No. 8,101,782, compounds which are hybrids of the carbamates rivastigmine and physostigmine may provide additive or synergistic therapeutic benefit, for example, for patients with Alzheimer's disease, Parkinson's disease, glaucoma, oncologic condition(s), or delayed gastric emptying, or patients suffering from attention deficit hyperactivity disorder (ADHD), phobia, stroke, multiple sclerosis, sleep disorders, psychiatric disorders, pain, anticholinergic drug overdose, or tobacco dependence i.e., use of the compounds in patients attempting smoking cessation. Although many carbamates (e.g. eptastigmine, quilostigmine, phenserine, tolserine) have been tested for their anticholinesterase activity, few have been effective and safe enough for use in treatment of patients (e.g. rivastigmine). Šarka Štěpánková, Cholinesterases and Cholinesterase Inhibitors, Current Enzyme Inhibition, 2008, 4, 160-171.

Many of the above listed diseases are fatal using current medical practice. In none of these diseases is there any known, widely accepted therapy or treatment that can halt and/or reverse the aggregation of amyloid deposits and in many, diagnosis remains difficult. As such there remains an urgent need for treatments.

Early definitive AD diagnosis in the living brain is also urgently needed as it could greatly facilitate specific timely treatment of the disorder and the search for novel drugs to preempt progress of this disease. In central nervous system (CNS) radioligand development, rapid screening of lead radiotracer candidates in animal models is an essential component in establishing a radioligand's product profile, putting the most promising candidates forward for evaluation in human clinical trials. In Alzheimer's disease (AD), a number of molecular imaging agents have been developed and evaluated in humans including those that target Aβ and tau. However, in general, these agents lack specificity for AD as up to 30% of cognitively normal individuals have evidence of this pathology. As such, a definitive diagnosis of AD during life remains elusive.

Although radioligands have been developed to detect deposition of AP plaques in the brain, many cognitively normal individuals also exhibit AP plaque deposition giving this approach inherent disadvantages for definitive AD diagnosis during life. The association of BuChE with AP plaques appears to be a characteristic of AD. This has prompted the search for radioligands that target BuChE in association with AP plaques that accumulate in cortical grey matter, a region normally with very little of this enzyme activity. A number of BuChE radioligands have been synthesized and preliminary testing indicates that some such radioligands enter the brain and accumulate in regions known to contain BuChE. Radioligands targeting unusual BuChE activity in the brain may represent a means for early diagnosis and treatment monitoring of AD.

### SUMMARY OF THE INVENTION

Several classes of radioligands are disclosed for brain imaging that target butyrylcholinesterase. Such compounds are capable of being used as diagnostics for AD and other diseases in which alteration of quantities, location, or regulation of BuChE in brain may be diagnostic of a pathology. Such compounds have particular utility in treatment of Alzheimer's disease and other amyloid diseases.

The radioligands of the present invention can be used for brain imaging that targets BuChE. The synthesis and in vivo evaluation of six such BChE radioligands are described here, which include pyridones: i) (p-[¹²³I]iodophenyl)methyl 6-oxo-1H-pyridine-2-carboxylate (TRV7005), ii) (p-[¹²³I]iodophenyl)methyl 1-methyl-6-oxo-1H-pyridine-2-carboxylate (TRV7006), iii) (p-[¹²³I]iodophenyl)methyl 6-methoxy-2-pyridinecarboxylate (TRV7019).

In accordance with the above, the present invention provides compounds of Formula I:

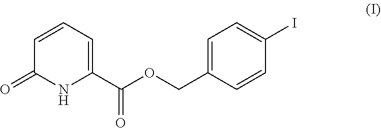 [SMILES: O=C(OCc1ccc(I)cc1)c1cccc(=O)[nH]1]

or a pharmaceutically acceptable salt thereof. The present invention also provides compounds of Formula II:

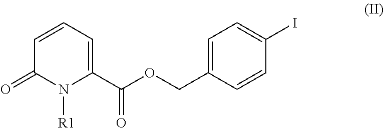

or a pharmaceutically acceptable salt thereof, in which R1 is alkyl, preferably methyl, ethyl, propyl, butyl, or pentyl. The present invention also provides compounds of Formula III:

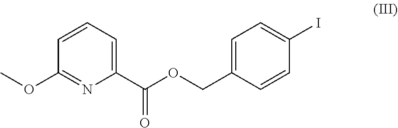 [SMILES: COc1cccc(C(=O)OCc2ccc(I)cc2)n1]

or a pharmaceutically acceptable salt thereof.

In each of Formulas I, II and III, “I” is iodine, and preferably ^{[123]}I for SPECT imaging.

The present invention also provides a method of diagnosis of Alzheimer's disease in a subject, including administering an effective amount of a compound of the present invention to the subject.

The present invention also provides a method of treatment of an amyloid disease in a subject, including administering an effective amount of a compound of the present invention to the subject.

The present invention also provides a method of diagnosis of multiple sclerosis in a subject, including administering an effective amount of a compound of the present invention to the subject.

The present invention also provides a method of diagnosis of brain tumour in a subject, including administering an effective amount of a compound of the present invention to the subject.

The present invention also provides a pharmaceutical composition having a compound of the present invention and a pharmaceutically acceptable excipient.

The present invention also provides a method for treating a condition which includes loss of memory, loss of cognition and a combination thereof, wherein the comprises administering to a subject in need thereof a therapeutically effective amount of a compound of Formula (I), Formula (II) or Formula (III) above. In certain embodiments, the condition is associated with Alzheimer's disease. The compound can be administered as a pharmaceutical composition comprising a pharmaceutically acceptable carrier. The total daily dose of the compound administered may be from about 0.0003 to about 30 mg/kg of body weight.

The present invention also provides a method of inhibiting butyrylcholinesterase activity in a patient which comprises administering to said patient a therapeutically effective amount of a compound of Formulas 1, II or III above.

The present invention also provides a method of treating a patient with Alzheimer's disease which comprises administering to said patient a therapeutically effective amount of a compound of Formula (I), Formula (II) or Formula (III) above.

The present invention also provides a method for treating an amyloid disease in a subject comprising administering to a subject in need thereof a therapeutically effective amount of a compound of Formulas I, II or III above. In certain embodiments, the amyloid disease may be Alzheimer's disease, Parkinson's disease or Huntington's disease.

In accordance with the above, the present invention is also directed to pharmaceutically acceptable salts, stereoisomers, polymorphs, metabolites, analogues, and pro-drugs of the compounds, and to any combination thereof.

With the foregoing and other advantages and features of the invention that will become hereafter apparent, the nature of the invention may be more clearly understood by reference to the following detailed description of the invention and the appended claims.

### BRIEF DESCRIPTION OF THE DRAWINGS

FIG. 1A shows the spectroscopic image for TRV 7005-I ¹H and FIG. 1B shows the spectroscopic image for TRV 7005-I ¹³C

FIG. 2A shows the spectroscopic image for TRV 7005-Sn ¹H and FIG. 2B shows the spectroscopic image for TRV 7005-Sn ¹³C

FIG. 3A shows the spectroscopic image for TRV 7006-I ¹H and FIG. 3B shows the spectroscopic image for TRV 7006413C

FIG. 4A shows the spectroscopic image for TRV7006-Sn ¹H and FIG. 4B shows the spectroscopic image for TRV7006-Sn ¹H

FIG. 5A shows the spectroscopic image for TRV 7019-I ¹H and FIG. 5B shows the spectroscopic image for TRV 7019-I ¹³C

FIG. 6A shows the spectroscopic image for TRV7019-Sn ¹H and FIG. 6B shows the spectroscopic image for TRV7019-Sn ¹³C

FIG. 7A shows the spectroscopic image for TRV 7040-I ¹H and FIG. 7B shows the spectroscopic image for TRV 7040-I ¹³C

FIG. 8A shows the spectroscopic image for TRV 7040-Sn ¹H and FIG. 8B shows the spectroscopic image for TRV 7040-Sn ¹³C

FIG. 9 shows radioligand concentration in the brain as a function of time, C(t), depicting the single-phase clearance of a radiotracer from the brain with an initial peak concentration, Cmax.

FIG. 10 is a TRV7005 dynamic planar single photon emission computed tomography (SPECT) scintigraphy scan for a representative 5XFAD mouse.

FIG. 11 is a TRV7005 dynamic planar single photon emission computed tomography (SPECT) scintigraphy scan for a representative WT mouse.

FIG. 12 shows whole brain time-activity curves for TRV7005 (expressed as percent of peak concentration (% Cₘₐₓ) in brain) with 12A showing mean time activity curves (mean±SEM) are shown for WT (green), 5XFAD (blue) and 5XFAD-BChE-KO (black); 12B showing time-activity curves having individual mice subject data and corresponding fitted exponential function (solid line); and 12C showing fitted exponential functions alone for individual mice from which kinetic summary measures were derived and compared.

FIG. 13 is a TRV7006 dynamic planar single photon emission computed tomography (SPECT) scintigraphy scan for a representative WT mouse.

FIG. 14 is a TRV7006 dynamic planar single photon emission computed tomography (SPECT) scintigraphy scan for a representative BChE-KO mouse.

FIG. 15 shows whole brain time-activity curves for TRV7006 (expressed as percent of peak concentration (% Cₘₐₓ) in brain) with 15A showing mean time activity curves (mean±SEM) are shown for WT (green) and BChE-KO (black); 15B showing time-activity curves having individual mice subject data and corresponding fitted exponential function (solid line); and 15C showing fitted exponential functions alone for individual mice from which kinetic summary measures were derived and compared.

FIG. 16 is a TRV7019 dynamic planar single photon emission computed tomography (SPECT) scintigraphy scan for a representative 5XFAD mouse.

FIG. 17 is a TRV7019 dynamic planar single photon emission computed tomography (SPECT) scintigraphy scan for a representative WT mouse.

FIG. 18 shows whole brain time-activity curves for TRV7019 (expressed as percent of peak concentration (% Cₘₐₓ) in brain) with 18A showing mean time activity curves (mean±SEM) are shown for WT (green) and 5XFAD (blue); 18B showing time-activity curves having individual mice subject data and corresponding fitted exponential function (solid line); and 18C showing fitted exponential functions alone for individual mice from which kinetic summary measures were derived and compared.

FIG. 19 is a TRV7040 dynamic planar single photon emission computed tomography (SPECT) scintigraphy scan for a representative 5XFAD mouse.

FIG. 20 is a TRV7040 dynamic planar single photon emission computed tomography (SPECT) scintigraphy scan for a representative WTmouse.

FIG. 22 shows whole brain time-activity curves for TRV7005, TRV7006, TRV7019, TRV5001 and TRV6001 radiotracers.

FIG. 23 shows kinetic summary measures of tracer clearance for TRV7005, TRV7006, TRV7019.

### DETAILED DESCRIPTION OF THE INVENTION

All patents, patent applications, and other publications referred to herein are hereby incorporated by reference in their entireties.

In one embodiment, compounds of Formula I are provided:

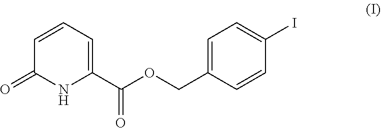 [SMILES: O=C(OCc1ccc(I)cc1)c1cccc(=O)[nH]1]

or a pharmaceutically acceptable salt thereof.

In one embodiment, compounds of Formula II are provided:

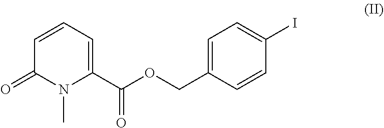 [SMILES: Cn1c(C(=O)OCc2ccc(I)cc2)cccc1=O]

or a pharmaceutically acceptable salt thereof

In one embodiment, compounds of Formula III are provided:

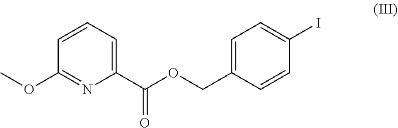 [SMILES: COc1cccc(C(=O)OCc2ccc(I)cc2)n1]

or a pharmaceutically acceptable salt thereof.

In one embodiment, a method of diagnosis of Alzheimer's disease in a subject is provided, comprising administering a diagnostically effective amount of a compound of the present invention to the subject.

In one embodiment, a pharmaceutical composition is provided comprising a compound of the present invention and a pharmaceutically acceptable excipient.

It is believed that the compounds of the invention bind to BuChE and modulate it thereby. Data supportive of this conclusion can be found in the Examples below.

### Definitions

Unless otherwise defined, terms as used in the specification refer to the following definitions, as detailed below.

The terms “administration” or “administering” compound should be understood to mean providing a compound of the present invention to an individual in a form that can be introduced into that individual's body in an amount effective for prophylaxis, treatment, or diagnosis, as applicable. Such forms may include e.g., oral dosage forms, injectable dosage forms, transdermal dosage forms, inhalation dosage forms, and rectal dosage forms.

The term “alkyl” as used herein means a straight or branched chain hydrocarbon containing from 1 to 20 carbon atoms, preferably from 1 to 10 carbon atoms, more preferably 1, 2, 3, 4, 5, or 6 carbons. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “carbonyl” as used herein means a —C(═O)— group.

The term “carboxy” as used herein means a —COOH group, which may be protected as an ester group: —COO-alkyl.

The compounds of the invention can be used in the form of pharmaceutically acceptable salts derived from inorganic or organic acids. Pharmaceutically acceptable salt(s) are well-known in the art. For clarity, the term “pharmaceutically acceptable salts” as used herein generally refers to salts prepared from pharmaceutically acceptable non-toxic acids or bases including inorganic acids and bases and organic acids and bases. Suitable pharmaceutically acceptable base addition salts include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art. See, e.g., Remington's Pharmaceutical Sciences, 18 th ed. (Mack Publishing, Easton Pa.: 1990) and Remington: The Science and Practice of Pharmacy, 19th ed. (Mack Publishing, Easton Pa.: 1995). The preparation and use of acid addition salts, carboxylate salts, amino acid addition salts, and zwitterion salts of compounds of the present invention may also be considered pharmaceutically acceptable if they are, within the scope of sound medical judgment, suitable for use in contact with the tissues of humans and lower animals without undue toxicity, irritation, allergic response, and the like, are commensurate with a reasonable benefit/risk ratio, and are effective for their intended use. Such salts may also include various solvates and hydrates of the compound of the present invention.

Certain compounds of the present invention may be isotopically labelled, e.g., with various isotopes of carbon, fluorine, or iodine, as applicable when the compound in question contains at least one such atom. In preferred embodiments, methods of diagnosis of the present invention comprise administration of such an isotopically labelled compound labeled with ¹²³I.

Certain compounds of the present invention may exist as stereoisomers wherein, asymmetric or chiral centers are present. These stereoisomers are “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The invention contemplates various stereoisomers and mixtures thereof and these are specifically included within the scope of this invention. Stereoisomers include enantiomers and diastereomers, and mixtures of enantiomers or diastereomers. Individual stereoisomers of compounds of the invention may be prepared synthetically from commercially available starting materials which contain asymmetric or chiral centers or by preparation of racemic mixtures followed by resolution well known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry”, 5th edition (1989), Longman Scientific & Technical, Essex CM20 2JE, England, or (2) direct separation of the mixture of optical enantiomers on chiral chromatographic columns or (3) fractional recrystallization methods.

Certain compounds of the present invention may exist as cis or trans isomers, wherein substituents on a ring may attach in such a manner that they are on the same side of the ring (cis) relative to each other, or on opposite sides of the ring relative to each other (trans). Such methods are well known to those of ordinary skill in the art, and may include separation of isomers by recrystallization or chromatography. It should be understood that the compounds of the invention may possess tautomeric forms, as well as geometric isomers, and that these also constitute an aspect of the invention.

It should be noted that a chemical moiety that forms part of a larger compound may be described herein using a name commonly accorded it when it exists as a single molecule or a name commonly accorded its radical. For example, the terms “pyridine” and “pyridyl” are accorded the same meaning when used to describe a moiety attached to other chemical moieties. Thus, for example, the two phrases “XOH, wherein X is pyridyl” and “XOH, wherein X is pyridine” are accorded the same meaning, and encompass the compounds pyridin-2-ol, pyridin-3-ol and pyridin-4-ol.

The term “pharmaceutically acceptable excipient”, as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of one skilled in the art of formulations.

Unless otherwise indicated, the terms “prevent,” “preventing” and “prevention” contemplate an action that occurs before a patient begins to suffer from the specified disease or disorder, which inhibits or reduces the severity of the disease or disorder or of one or more of its symptoms. The terms encompass prophylaxis.

Unless otherwise indicated, a “prophylactically effective amount” of a compound is an amount sufficient to prevent a disease or condition, or one or more symptoms associated with the disease or condition, or prevent its recurrence. A prophylactically effective amount of a compound is an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

Unless otherwise indicated, a “diagnostically effective amount” of a compound is an amount sufficient to diagnose a disease or condition. In general, administration of a compound for diagnostic purposes does not continue for as long as a therapeutic use of a compound, and could be administered only once if such is sufficient to produce the diagnosis.

Unless otherwise indicated, a “therapeutically effective amount” of a compound is an amount sufficient to treat a disease or condition, or one or more symptoms associated with the disease or condition.

The term “subject” is intended to include living organisms in which disease may occur. Examples of subjects include humans, monkeys, cows, sheep, goats, dogs, cats, mice, rats, and transgenic species thereof.

The term “substantially pure” means that the isolated material is at least 90% pure, preferably 95% pure, even more preferably 99% pure as assayed by analytical techniques known in the art.

The pharmaceutical compositions can be formulated for oral administration in solid or liquid form, for parenteral intravenous, subcutaneous, intramuscular, intraperitoneal, intra-arterial, or intradermal injection, for or for vaginal, nasal, topical, or rectal administration. Pharmaceutical compositions of the present invention suitable for oral administration can be presented as discrete dosage forms, e.g., tablets, chewable tablets, caplets, capsules, liquids, and flavored syrups. Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Parenteral dosage forms can be administered to patients by various routes including subcutaneous, intravenous (including bolus injection), intramuscular, and intraarterial. Because their administration typically bypasses patients' natural defenses against contaminants, parenteral dosage forms are specifically sterile or capable of being sterilized prior to administration to a patient. Examples of parenteral dosage forms include solutions ready for injection, dry products ready to be dissolved or suspended in a pharmaceutically acceptable vehicle for injection, suspensions ready for injection, and emulsions. Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like, and suitable mixtures thereof), vegetable oils (such as olive oil) and injectable organic esters such as ethyl oleate, or suitable mixtures thereof. Suitable fluidity of the composition may be maintained, for example, by the use of a coating such as lecithin, by the maintenance of the required particle size in the case of dispersions, and by the use of surfactants. These compositions may also contain adjuvants such as preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form may be brought about by the use of agents delaying absorption, for example, aluminum monostearate and gelatin.

In some cases, in order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Suspensions, in addition to the active compounds, may contain suspending agents, for example, ethoxylated isostearyl alcohols, polyoxyethylene sorbitol and sorbitan esters, microcrystalline cellulose, aluminum metahydroxide, bentonite, agar-agar, tragacanth, and mixtures thereof. If desired, and for more effective distribution, the compounds of the invention can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations also are prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Solid dosage forms for oral administration include capsules, tablets, pills, powders, and granules. In such solid dosage forms, one or more compounds of the invention is mixed with at least one inert pharmaceutically acceptable carrier such as sodium citrate or dicalcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using lactose or milk sugar as well as high molecular weight polyethylene glycols. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract in a delayed manner. Examples of materials which can be useful for delaying release of the active agent can include polymeric substances and waxes.

Compositions for rectal or vaginal administration are preferably suppositories which can be prepared by mixing the compounds of this invention with suitable non-irritating carriers such as cocoa butter, polyethylene glycol or a suppository wax which are solid at ambient temperature but liquid at body temperature and therefore melt in the rectum or vaginal cavity and release the active compound.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents. Dosage forms for topical or transdermal administration of a compound of this invention include ointments, pastes, creams, lotions, gels, powders, solutions, sprays, inhalants or patches. A desired compound of the invention is admixed under sterile conditions with a pharmaceutically acceptable carrier and any needed preservatives or buffers as may be required. Ophthalmic formulation, ear drops, eye ointments, powders and solutions are also contemplated as being within the scope of this invention. The ointments, pastes, creams and gels may contain, in addition to an active compound of this invention, animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, lactose, talc, silicic acid, aluminum hydroxide, calcium silicates and polyamide powder, or mixtures of these substances. Sprays can additionally contain customary propellants such as chlorofluorohydrocarbons.

Compounds of the invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds of the invention, stabilizers, preservatives, and the like. The preferred lipids are the natural and synthetic phospholipids and phosphatidylcholines (lecithins) used separately or together. Methods to form liposomes are known in the art. See, for example, Prescott, Ed., Methods in Cell Biology, Volume XIV, Academic Press, New York, N.Y., (1976), p 33 et seq.

Actual dosage levels of active ingredients in the pharmaceutical compositions of this invention can be varied so as to obtain an amount of the active compound(s) that is effective to achieve the desired therapeutic response for a particular patient, compositions and mode of administration. The selected dosage level will depend upon the activity of the particular compound, the route of administration, the severity of the condition being treated and the condition and prior medical history of the patient being treated. However, it is within the skill of the art to start doses of the compound at levels lower than required to achieve the desired therapeutic effect and to gradually increase the dosage until the desired effect is achieved.

An effective amount of one of the compounds of the invention can be employed in pure form or, where such forms exist, in pharmaceutically acceptable salt form. Alternatively, the compound can be administered as a pharmaceutical composition containing the compound of interest in combination with one or more pharmaceutically acceptable carriers. It will be understood, however, that the total daily usage of the compounds and compositions of the invention will be decided by the attending physician within the scope of sound medical judgment. The specific effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; the risk/benefit ratio; drugs used in combination or coincidental with the specific compound employed; and like factors well known in the medical arts. For example, it is well within the skill of the art to start doses of the compound at levels lower than required to achieve the desired therapeutic effect and to gradually increase the dosage until the desired effect is achieved.

The total daily dose of the compounds of the present invention as administered to a human or lower animal may range from about 0.0003 to about 30 mg/kg of body weight. For purposes of oral administration, more preferable doses can be in the range of from about 0.0003 to about 1 mg/kg body weight. If desired, the effective daily dose can be divided into multiple doses for purposes of administration; consequently, single dose compositions may contain such amounts or submultiples thereof to make up the daily dose. For oral administration, the compositions of the invention are preferably provided in the form of tablets containing about 1.0, about 5.0, about 10.0, about 15.0, about 25.0, about 50.0, about 100, about 250, or about 500 milligrams of the active ingredient.

Diagnostic uses can be as probes which, in conjunction with non-invasive neuroimaging techniques such as magnetic resonance spectroscopy (MRS) or imaging (MRI), or gamma imaging such as positron emission tomography (PET) or single-photon emission computed tomography (SPECT), are used to identify neuritic plaques (NP). For in vivo imaging, detection instrument availability greatly affects selection of a given label. The type of instrument used will guide the selection of the radionuclide or stable isotope. For instance, the radionuclide chosen must have a type of decay detectable by a given type of instrument. Another consideration relates to the half-life of the radionuclide. The half-life should be long enough so that it is still detectable at the time of maximum uptake by the target, but short enough so that the host does not sustain deleterious radiation. The radiolabeled compounds of the present invention can be detected using gamma imaging wherein emitted gamma irradiation of the appropriate wavelength is detected. Methods of gamma imaging include, but are not limited to, SPECT and PET. After a sufficient time has elapsed for the compound to bind BuChE (in a range between 30 minutes and 48 hours, for example), the area of the subject under investigation is examined by routine imaging techniques such as MRS/MRI, SPECT, PET, and CT. The exact protocol will necessarily vary depending upon factors specific to the patient, as noted above, and depending upon the body site under examination, method of administration and type of label used.

Radiolabelled diagnostics, using e.g. both human postmortem brain tissues as well as mouse animal model of Alzheimer's disease, can also be used as an in vitro methodology for rapidly screening for compounds that can detect butyrylcholinesterase activity associated with Alzheimer's disease using autoradiography as a specific in vitro screening system.

The following is a method of the present invention for the production of radioligands. Compounds with a leaving group such as a tributyl tin, triflates or tosylates are dissolved in an appropriate solvent. To exchange the leaving group for iodine, the compound is treated with the appropriate reagent to incorporate radio-iodide. The exchange for fluorine is performed using potassium fluoride. These reactions are carried out until the starting material has disappeared using TLC analysis. The solvent is then evaporated and the product dissolved in dichloromethane or methanol. The product is purified by SEP pak and/or HPLC. Radio-iodination involves substitution of the precursor with an appropriate leaving group. The chemical reagent grade radionuclides are commercially available (¹²³I NaI, ¹³¹I NaI) as sodium iodide in sodium hydroxide solution. Precursors for radio-iodination include molecules with leaving groups such as tributyl tin, triflate and tosylate derivatives. The radiolabeled molecules are meant to be used for enzymatic assessment and binding assays. ¹²³I Labeling may be performed using N-chlorosuccinimide, iodobead or iodogen as a free radical initiator. The precursor is dissolved in an appropriate solvent and incubated with ¹²³I sodium iodide.

### EXAMPLES

### Synthetic Methods

All non-aqueous reactions were carried out in flame-dried round bottom flasks under an inert atmosphere (nitrogen or argon), unless otherwise stated. Temperatures indicated refer to an external bath. All reactions were magnetically stirred.

All organic solvents were distilled and dried following known procedures. Reagents were purchased from various commercial suppliers and used without further purification. The starting materials are either commercially available or may be prepared from commercially available reagents using chemical reactions known in the art.

### TRV7005-I Synthetic Procedure and Spectroscopic Data

### Example 1

### (p-Iodophenyl)methyl 6-oxo-1H-pyridine-2-carboxylate (TRV7005-I)

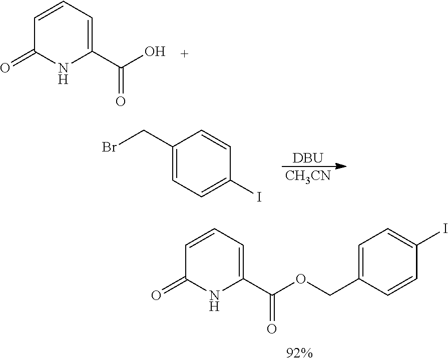

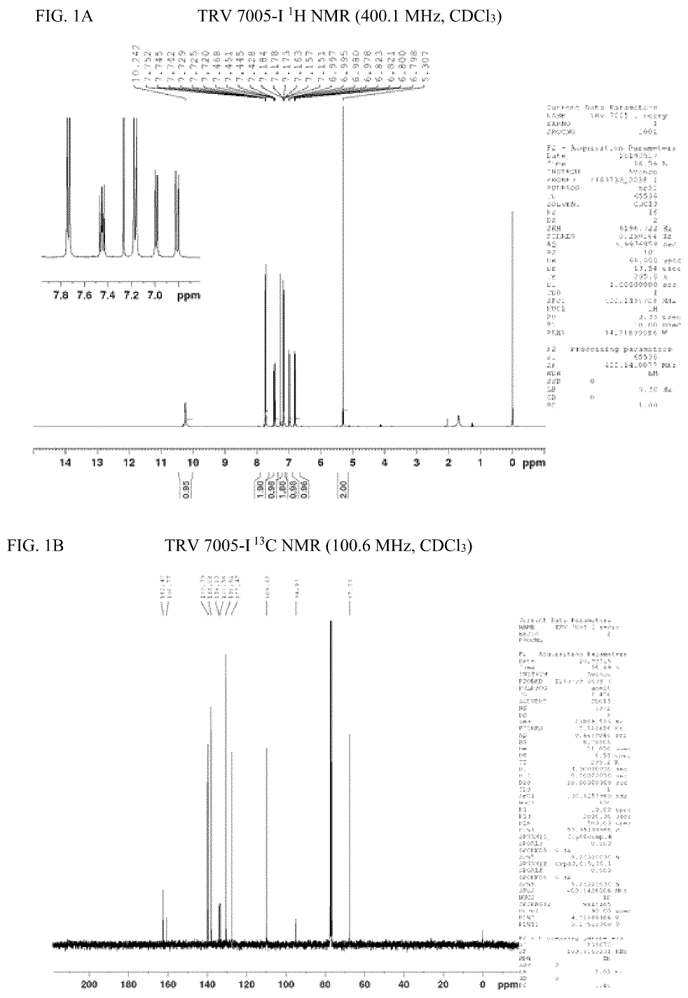

6-Hydroxypyridine-2-carboxylic acid (1.423 g, 10.2 mmol) was placed in acetonitrile (15 mL) in an RBF (50 mL). 1,8-Diazabicyclo[5.4.0]undec-7-ene (1.53 mL, 10.2 mmol) was added dropwise to the mixture. 4-Iodobenyl bromide (3.0365 g, 10.2 mol) was added the reaction as a solid and the resulting mixture was stirred for 16 hr at room temperature. After this time requirement, the reaction was poured into water and this was extracted with dichloromethane (5×50 mL). The combined organic layers were dried over Na₂SO₄, gravity filtered and concentrated in vacuo affording a spectroscopically clean (p-Iodophenyl)methyl 6-oxo-1H-pyridine-2-carboxylate (3.326 g, 92%). The Spectroscopic images are found in FIGS. 1A and 1B. mp: 177-179° C.; IR (ATR) 3031, 2928, 1740, 1655, 1607, 1280, 1258, 1142, 1001, 803, 756 cm⁻¹; ¹H NMR (400.1 MHz, CDCl₃) δ 10.24 (s, 1H), 7.75-7.72 (m, 2H), 7.45 (dd, J=9.3, 6.8 Hz, 1H), 7.18-7.15 (m, 2H), 6.99 (dd, J=6.7, 0.8 Hz, 1H) 6.81 (dd, J=9.3, 0.8 Hz, 1H) 5.30 (s, 2H); ¹³C NMR (100.6 MHz, CDCl₃) δ 162.5 (C11), 160.8 (C6), 139.8 (C9), 138.1 (C2), 134.2 (C4), 133.6 (C7), 130.6 (C3), 127.5 (C10), 109.8 (C8), 95.0 (C1), 67.8 (C5).

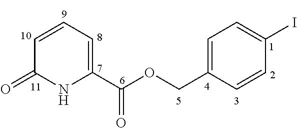 [SMILES: O=C(OCc1ccc(I)cc1)c1cccc(=O)[nH]1]

### Example 2

### [p-(tributylstannyl)phenyl]methyl 6-oxo-1H-pyridine-2-carboxylate (TRV7005-Sn)

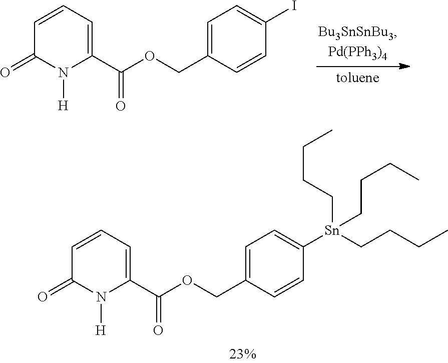 [SMILES: O=C(OCc1ccc(I)cc1)c1cccc(=O)[nH]1 ; CCC[CH2][Sn]([CH2]CCC)([CH2]CCC)[c]1ccc(COC(=O)c2cccc(=O)[nH]2)cc1]

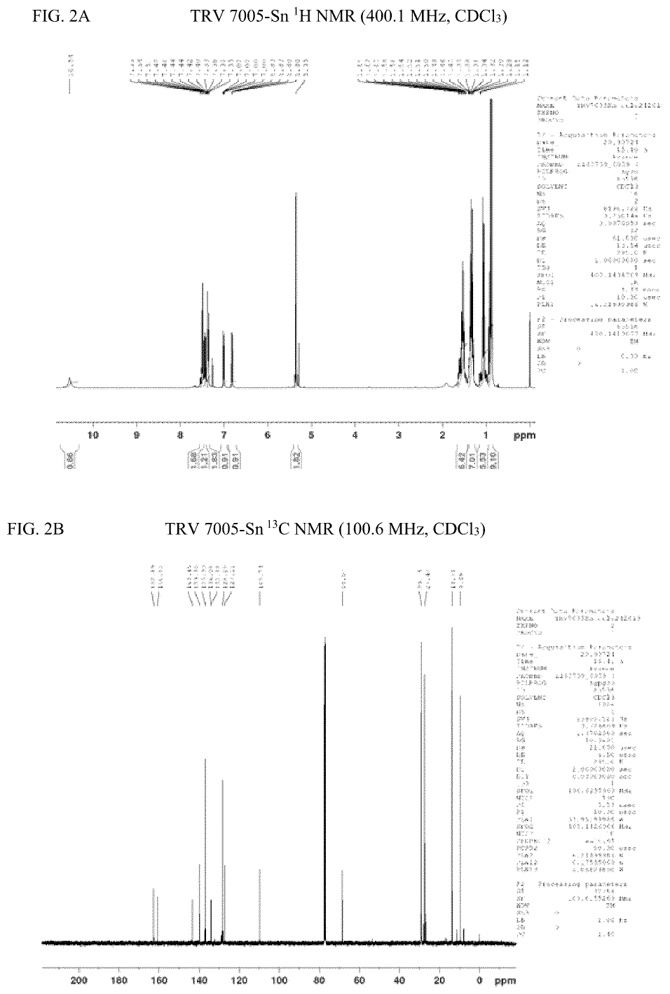

To degassed toluene (25 mL) and under an atmosphere of anhydrous argon, (p-Iodophenyl)methyl 6-oxo-1H-pyridine-2-carboxylate (0.385 g, 1.08 mmol), tetrakistriphenylphosphine palladium(0) (0.0400 g, 0.0346 mmol), and hexabutylditin (0.942 g, 1.62 mmol) were added sequentially. The resulting reaction mixture was heated to reflux temperature for 24 hrs. After this time, the mixture was cooled to room temperature, concentrated in vacuo and purified using flash chromatography through silica gel (30% EtOAc/70% CH2Cl2) to afford a clear light grey liquid (0.120 g, 23%). The Spectroscopic images are found in FIGS. 2A and 2B. 1H NMR (400.1 MHz, CDCl₃) δ 10.54 (s, 1H), 7.55-7.49 (m, 2H), 7.44 (dd, J=9.3, 6.8 Hz, 1H), 7.40-7.35 (m, 2H), 7.01 (dd, J=6.8, 0.9 Hz, 1H), 6.81 (dd, J=9.3, 0.9 Hz, 1H), 5.35 (s, 2H), 1.64-1.46 (m, 6H), 1.40-1.28 (m, 6H), 1.15-0.98 (m, 6H), 0.89 (t, J=7.3 Hz, 9H); 13C NMR (100.6 MHz, CDCl₃) δ 162.7 (C11), 160.8 (C6), 143.5 (1C), 139.8 (C9), 136.9 (C2), 134.1 (C4), 133.9 (C7), 128.2 (C3), 127.2 (C10), 109.7 (C8), 68.6 (C5), 29.2 (C9), 27.5 (C14), 13.8 (C15), 9.7 (C12).

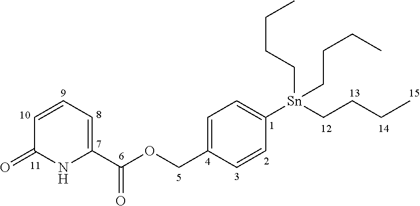 [SMILES: CCC[CH2][Sn]([CH2]CCC)([CH2]CCC)[c]1ccc(COC(=O)c2cccc(=O)[nH]2)cc1]

### Example 3

### (p-Iodophenyl)methyl 1-methyl-6-oxo-1H-pyridine-2-carboxylate (TRV7006-I)

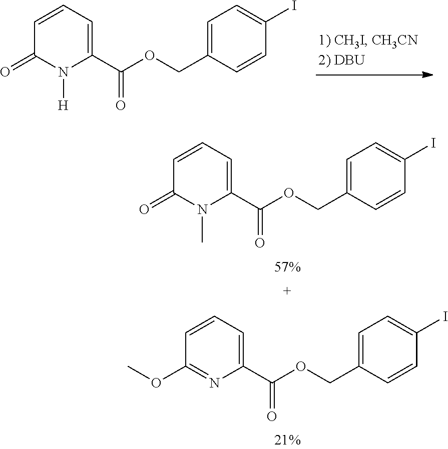 [SMILES: O=C(OCc1ccc(I)cc1)c1cccc(=O)[nH]1 ; Cn1c(C(=O)OCc2ccc(I)cc2)cccc1=O ; COc1cccc(C(=O)OCc2ccc(I)cc2)n1]

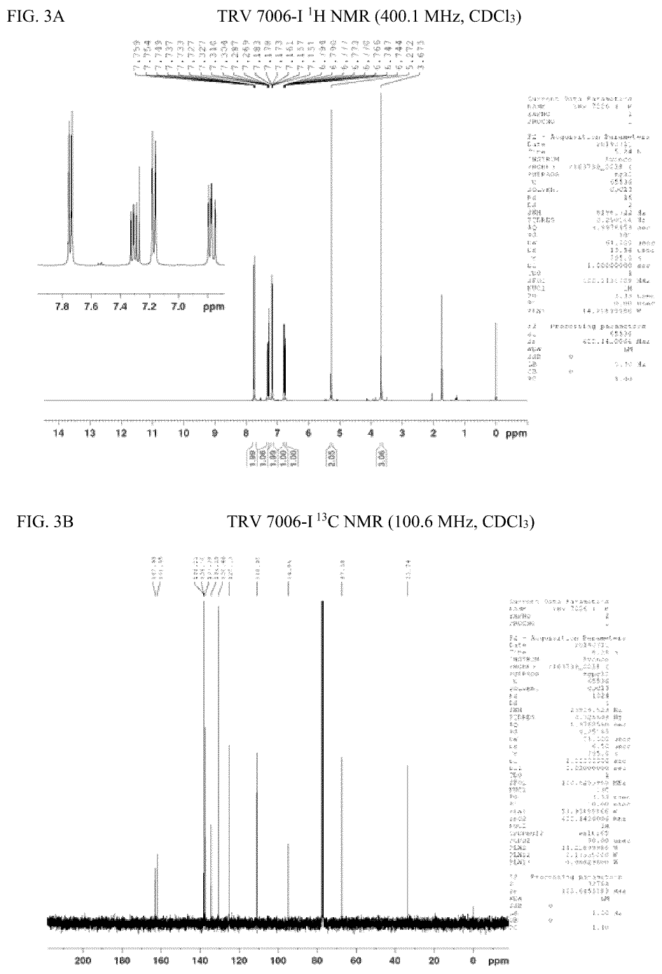

(p-Iodophenyl)methyl 6-oxo-1H-pyridine-2-carboxylate (0.500 g, 1.41 mmol) was placed in acetonitrile (5 mL) within a reaction vial (20 mL). To this mixture, methyl iodide (0.099 mL, 1.6 mmol) and DBU (0.211 mL, 1.41 mmol) were sequentially added. The resulting mixture was stirred at rt for 36 hours. After this time, the reaction was then poured into water (30 mL) and extracted with dichloromethane (3×20 mL). The combined organic layers were dried over Na₂SO₄, gravity filtered and evaporated under vacuum affording a white solid. This solid was purified using flash chromatography through silica gel (1% MeOH/99% CH₂Cl₂) to afford (p-Iodophenyl)methyl 1-methyl-6-oxo-1H-pyridine-2-carboxylate (0.2964 g, 57%) and (p-Iodophenyl)methyl 6-methoxy-2-pyridinecarboxylate (0.1095 g, 21%). The Spectroscopic images are found in FIGS. 3A and 3B. : mp: 83-85° C.; IR (ATR) 3031, 2951, 1727, 1654, 1587, 1246, 1078, 795, 764 cm⁻¹; ¹H NMR (400.1 MHz, CDCl₃) δ 7.76-7.73 (m, 2H), 7.31 (dd, J=9.1, 6.9 Hz, 1H), 7.18-7.15 (m, 2H), 6.78 (dd, J=6.9, 1.4 Hz, 1H), 6.76 (dd, J=9.2, 1.4 Hz, 1H), 5.27 (s, 2H), 3.67 (s, 3H); ¹³C NMR (100.6 MHz, CDCl₃) δ 163.0(C11), 162.0(C5), 138.2(C7), 138.1(C2), 137.4(C9), 134.4(C4), 130.5(C3), 125.1(C10), 110.1(C8), 94.9(C1), 67.4(C5), 33.7(C12); LRMS(EI, 70 eV): 370(M⁺¹, 5), 369(M⁺, 31), 217(100), 152(21), 136(5), 122(5), 108(12), 90(26), 89(18); HPLC(90% CH₃CN/10% H₂O, 1 mL/min): 1.877 min, 100%.

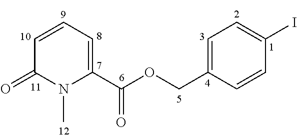 [SMILES: Cn1c(C(=O)OCc2ccc(I)cc2)cccc1=O]

### Example 4

### [p-(tributylstannyl)phenyl]methyl 1-methyl-6-oxo-1H-pyridine-2-carboxylate (TRV7006-Sn)

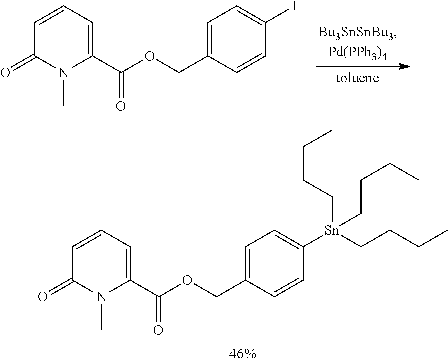 [SMILES: Cn1c(C(=O)OCc2ccc(I)cc2)cccc1=O ; CCC[CH2][Sn]([CH2]CCC)([CH2]CCC)[c]1ccc(COC(=O)c2cccc(=O)n2C)cc1]

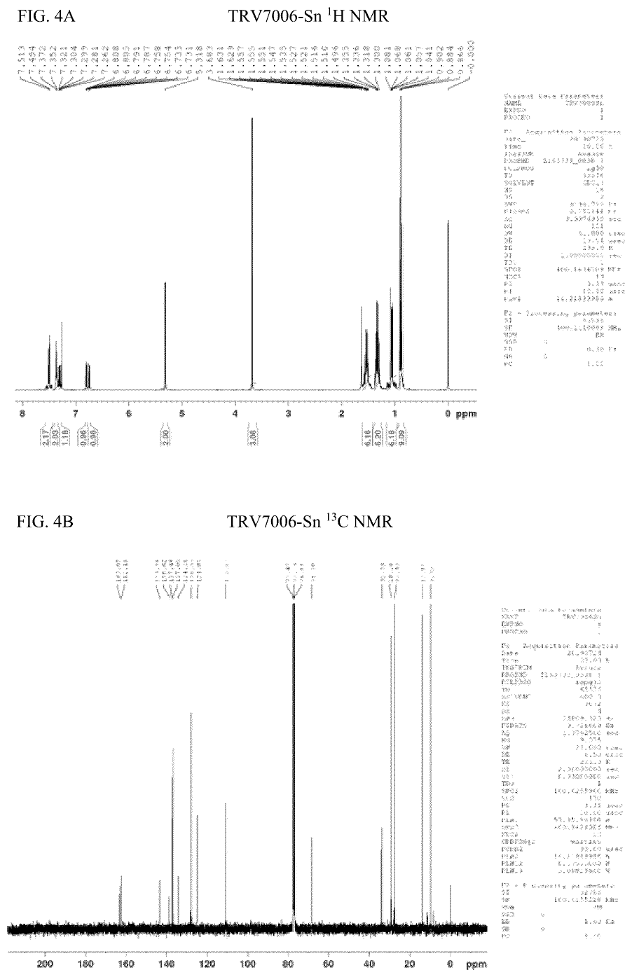

degassed toluene (25 mL) and under an atmosphere of anhydrous argon, (p-Iodophenyl)methyl 1-methyl-6-oxo-1H-pyridine-2-carboxylate (0.292 g, 0.790 mmol), tetrakistriphenylphosphine palladium(0) (0.0282 g, 0.0244 mmol), and hexabutylditin (0.687 g, 1.18 mmol) were added sequentially. The resulting reaction mixture was heated to reflux temperature for 24 hrs. After this time, the mixture was cooled to room temperature, concentrated in vacuo and purified using flash chromatography through silica gel (CH₂Cl₂) to afford a clear liquid (0.193 g, 46%). Viscous liquid The Spectroscopic images are found in FIGS. 4A and 4B. 1H NMR (400.1 MHz, CDCl₃) δ 7.56-7.45 (m, 2H), 7.38-7.34 (m, 2H), 7.30 (dd, J=9.2, 6.9 Hz, 1H), 6.80 (dd, J=6.9, 1.4 Hz, 1H), 6.74 (dd, J=6.7, 1.4 Hz, 1H), 5.32 (s, 2H), 3.68 (s, 3H), 1.60-1.48 (m, 6H), 1.37-1.28 (m, 6H), 1.15-1.00 (m, 6H), 0.88 (t, J=7.3 Hz, 9H); ¹³C NMR (100.6 MHz, CDCl₃) δ 163.1 (C11), 162.2 (C6), 143.5 (C1), 138.9 (C7), 137.5 (C9), 137.0 (C2), 134.3 (C4), 128.1 (C3), 124.9 (C10), 110.8 (C8), 68.3 (C5), 33.8 (C12), 29.2 (C15), 27.5 (C14), 13.8 (C16), 9.7 (C13).

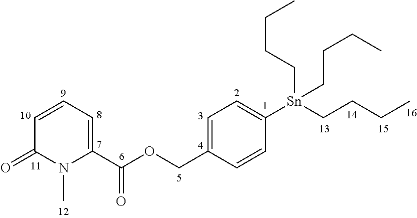 [SMILES: CCC[CH2][Sn]([CH2]CCC)([CH2]CCC)[c]1ccc(COC(=O)c2cccc(=O)n2C)cc1]

### Example 5

### (p-iodophenyl)methyl 6-methoxy-2-pyridinecarboxylate (TRV7019)

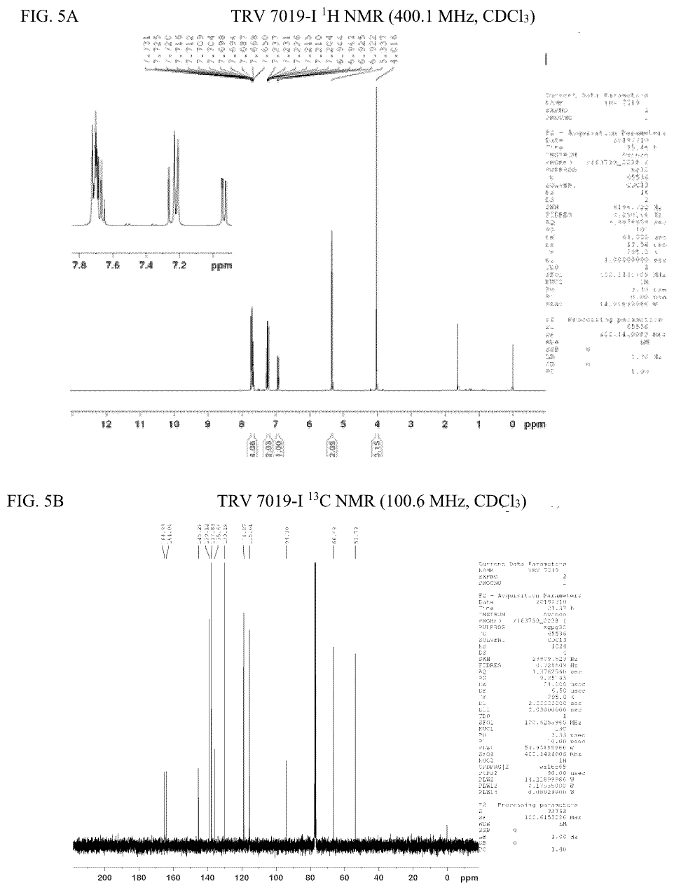

mmol) was placed in acetonitrile (5 mL) within a reaction vial (20 mL). To this mixture, methyl iodide (0.099 mL, 1.6 mmol) and DBU (0.211 mL, 1.41 mmol) were sequentially added. The resulting mixture was stirred at rt for 36 hours. After this time, the reaction was then poured into water (30 mL) and extracted with dichloromethane (3×20 mL). The combined organic layers were dried over Na₂SO₄, gravity filtered and evaporated under vacuum affording a white solid. This solid was purified using flash chromatography through silica gel (1% MeOH/99% CH₂Cl₂) to afford (p-Iodophenyl)methyl 1-methyl-6-oxo-1H-pyridine-2-carboxylate (0.2964 g, 57%) and (p-Iodophenyl)methyl 6-methoxy-2-pyridinecarboxylate (0.1095 g, 21%). The Spectroscopic images are found in FIGS. 5A and 5B. mp: 76-79° C.; IR (ATR) 3008, 2986, 2950, 1732, 1592, 1466, 1365, 1244, 1147, 797, 768 cm⁻¹; ¹H NMR (400.1 MHz, CDCl₃) δ 7.73-7.65 (m, 4H), 7.24-7.20 (m, 2H), 6.93 (dd, J=7.8, 1.3 Hz, 1H), 5.34 (s, 2H), 4.01 (s, 3H); ¹³C NMR (100.6 MHz, CDCl₃) δ 165.0(6C), 164.1(C11), 145.3(C7), 139.1(C9), 137.8(C2), 135.6(C4), 130.2(C3), 118.9(C8), 115.6(C10), 94.1(C1), 66.5(C5), 53.8(C12).

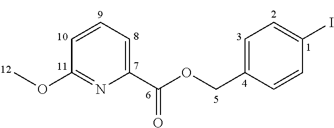 [SMILES: COc1cccc(C(=O)OCc2ccc(I)cc2)n1]

### Example 6

### [p-(tributylstannyl)phenyl]methyl 6-methoxy-2-pyridinecarboxylate (TRV7019-Sn)

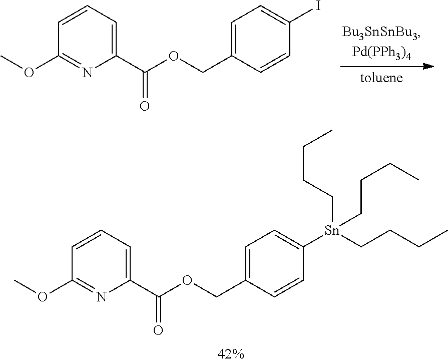 [SMILES: COc1cccc(C(=O)OCc2ccc(I)cc2)n1 ; CCC[CH2][Sn]([CH2]CCC)([CH2]CCC)[c]1ccc(COC(=O)c2cccc(OC)n2)cc1]

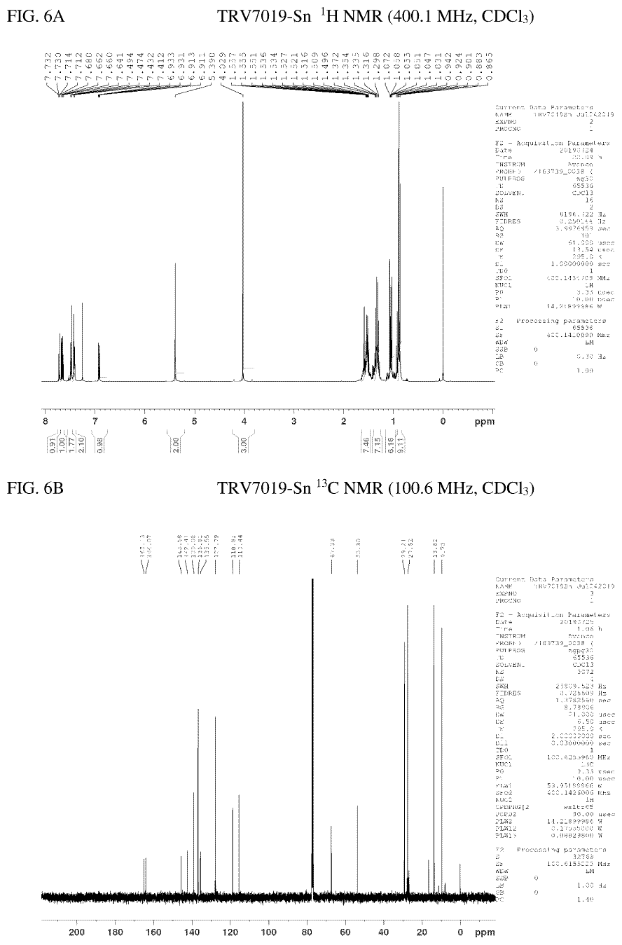

To degassed toluene (25 mL) and under an atmosphere of anhydrous argon, (p-Iodophenyl)methyl 6-methoxy-2-pyridinecarboxylate (0.221 g, 0.660 mmol), tetrakistriphenylphosphine palladium(0) (0.0236 g, 0.0204 mmol), and hexabutylditin (0.1.18 g, 2.04 mmol) were added sequentially. The resulting reaction mixture was heated to reflux temperature for 24 hrs. After this time, the mixture was cooled to room temperature, concentrated in vacuo and purified using flash chromatography through silica gel (CH₂Cl₂) to afford a clear liquid (0.148 g, 42%). The Spectroscopic images are found in FIGS. 6A and 6B. 1H NMR (400.1 MHz, CDCl₃) δ 7.72 (dd, J=7.3, 1.0 Hz, 1H), 7.66 (dd, J=8.1, 7.3 Hz, 1H), 7.54-7.47 (m, 2H), 7.44-7.40 (m, 2H), 6.92 (dd, J=8.1, 1.0 Hz, 1H), 5.39 (s, 2H), 4.03 (s, 3H), 1.65-1.45 (m, 6H), 1.42-1.28 (m, 6H), 1.14-0.97 (m, 6H), 0.88 (t, J=7.3 Hz, 9H); ¹³C NMR (100.6 MHz, CDCl₃) δ 165.1 (C6), 164.1 (C11), 145.6 (C7), 142.4 (C1), 139.1 (C9), 136.8 (C2), 135.6 (C4), 127.8 (C3), 118.9 (C8), 115.4 (C10), 67.3 (C5), 53.8 (C12), 29.2 (C14), 27.5 (C15), 13.8 (C16), 9.7 (C13).

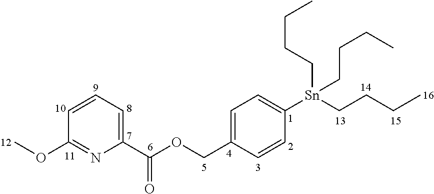 [SMILES: CCC[CH2][Sn]([CH2]CCC)([CH2]CCC)[c]1ccc(COC(=O)c2cccc(OC)n2)cc1]

### Example 7

### Benzyl 6-[(p-iodophenyl)methoxy]-2-pyridinecarboxylate (TRV7040-I)

### Step 1

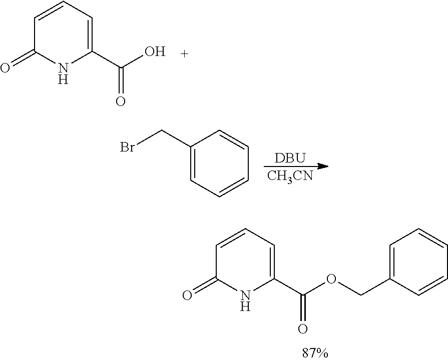

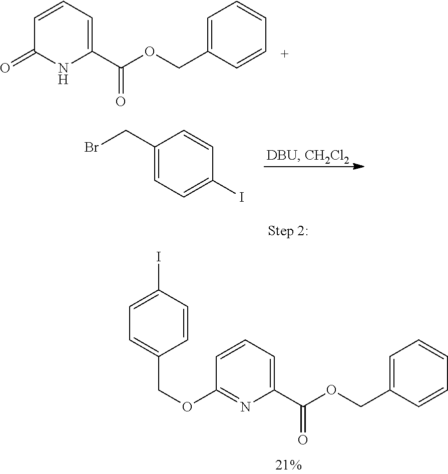

Step 1: 6-Hydroxypyridine-2-carboxylic acid (5.020 g, 36.09 mmol) was placed in acetonitrile (30 mL) in a RBF (100 mL). 1,8-Diazabicyclo[5.4.0]undec-7-ene (5.26 mL, 35.22 mmol) was added dropwise to the mixture. Benyl bromide (6.147 g, 35.94 mmol) was added the reaction as a solid and the resulting mixture was stirred for 24 hr at room temperature. After this time requirement, the reaction was poured into water (50 mL) and this was extracted with dichloromethane (2×50 mL). The combined organic layers were dried over Na₂SO₄, gravity filtered and concentrated in vacuo affording Benzyl 6-oxo-1H-pyridine-2-carboxylate (7.142 g, 87%).

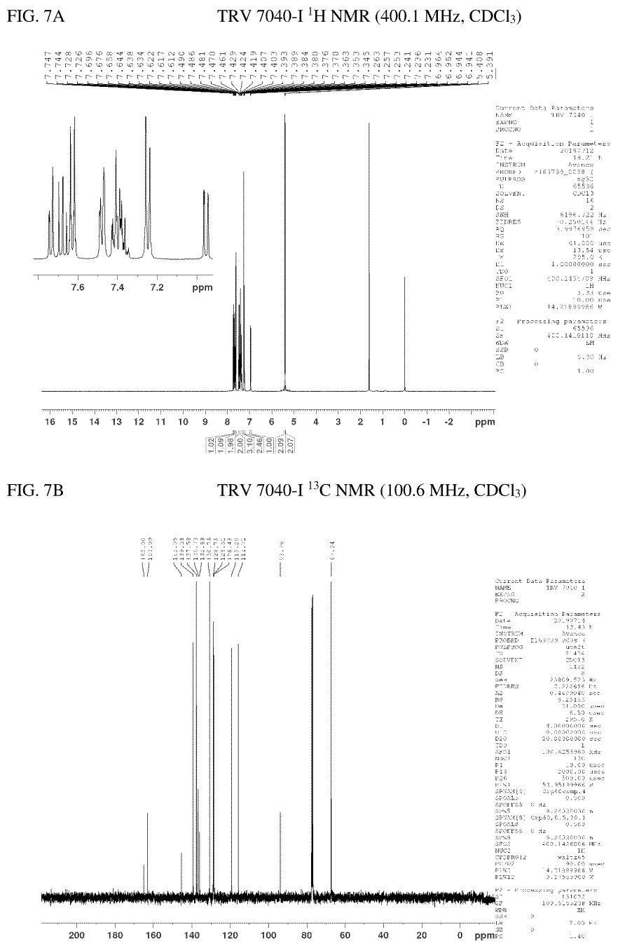

Step 2: Benzyl 6-oxo-1H-pyridine-2-carboxylate (7.000 g, 30.54 mmol) was dissolved in dichloromethane (30 mL) with in a RBF (100 mL). 4-Iodobenzyl bromide (9.068 g, 30.54 mmol) was added to the solution and stirred for 20 minutes. To this mixture, DBU (4.649 g, 30.54 mmol) was added and stirred for 30 hrs. After this time requirement, reaction was poured into water (50 mL) and extracted with dichloromethane (2×50 mL). The combined organic layers were dried over MgSO4, gravity filtered, and the filtrate was concentrated in vacuo. The resulting crude material was placed in hexanes (50 mL) and vigorously stirred for 10 min and the solid was collected by suction filtration. This hexane procedure was repeated two more times. The isolated solid from the last suction filtration was purified using flash chromatography (50% hexane/50% dichloromethane) to afford benzyl 6-[(p-iodophenyl)methoxy]-2-pyridinecarboxylate (2.9107 g, 21%). The Spectroscopic images are found in FIGS. 7A and 7B. mp: 82-82° C.; IR (ATR) 3088, 3056, 3035, 1727, 1596, 1447, 1264, 1246, 1021, 760, 731 cm⁻¹; ¹H NMR (400.1 MHz, CDCl₃) δ 7.74 (dd, J=7.3, 0.9 Hz, 1H), 7.68 (dd, J=8.1, 7.3 Hz, 1H), 7.64-7.61 (m, 2H), 7.49-7.46 (m, 2H), 7.44-7.34 (m, 3H), 7.26-7.23 (m, 2H), 6.95 (dd, J=8.1, 0.9 Hz, 1H), 5.41 (s, 2H), 5.39 (s, 2H); ¹³C NMR (100.6 MHz, CDCl₃) δ165.0(C6), 163.1(C11), 145.3(C7), 139.4(C9), 137.6(C15), 136.7(C13), 135.9(C4), 130.8(C14), 128.8(C2), 128.5(C1), 128.5(C3), 119.2(C8), 115.7(C10), 93.8(C16), 67.3(C5, C16).

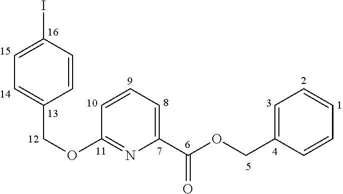 [SMILES: O=C(OCc1ccccc1)c1cccc(OCc2ccc(I)cc2)n1]

### Example 8

### Benzyl 6-[(p-(tributylstannyl)phenyl)methoxy]-2-pyridinecarboxylate TRV7040-Sn

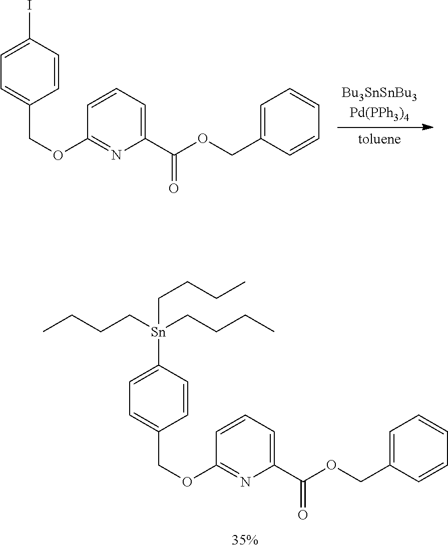 [SMILES: O=C(OCc1ccccc1)c1cccc(OCc2ccc(I)cc2)n1 ; CCC[CH2][Sn]([CH2]CCC)([CH2]CCC)[c]1ccc(COc2cccc(C(=O)OCc3ccccc3)n2)cc1]

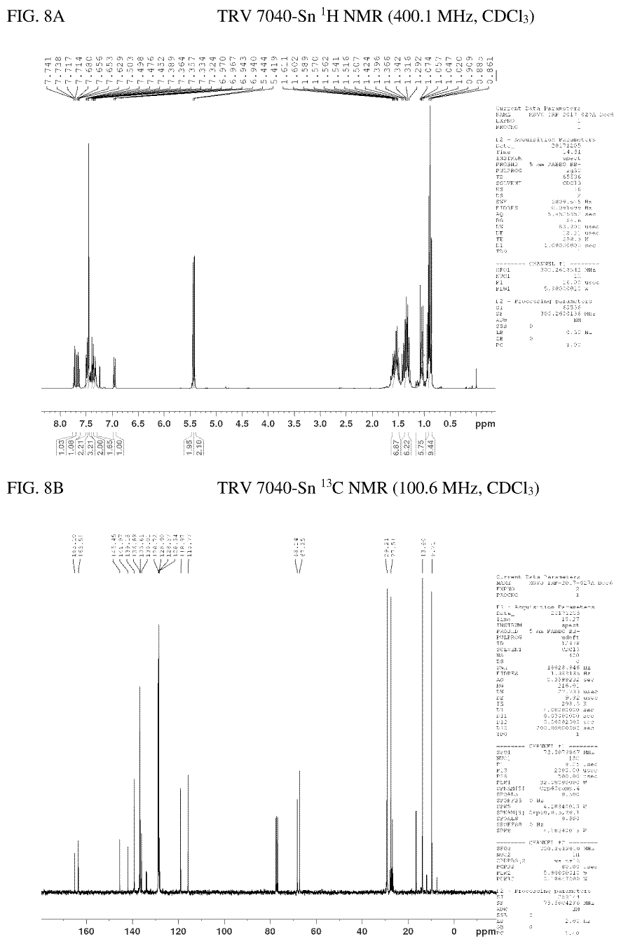

To degassed toluene (15 mL) and under an atmosphere of anhydrous argon, benzyl 6-[(p-iodophenyl)methoxy]-2-pyridinecarboxylate (0.151 g, 0.339 mmol), tetrakistriphenylphosphine palladium(0) (0.0196 g, 0.0169 mmol), and hexabutylditin (1.14 g, 508 mmol) were added sequentially. The resulting reaction mixture was heated to reflux temperature for 24 hrs. After this time, the mixture was cooled to room temperature, concentrated in vacuo and purified using flash chromatography through silica gel (10% ethyl acetate/90% hexane) to afford a clear liquid (0.0713 g, 35%). The Spectroscopic images are found in FIGS. 8A and 8B. 1H NMR (400.1 MHz, CDCl₃) δ 7.73 (dd, J=7.3, 1.0 Hz, 1H), 7.65 (dd, J=8.1, 7.3 Hz, 1H), 7.50-7.31 (m, 9H), 6.95 (dd, J=8.1, 1.0 Hz, 1H), 5.44 (s, 2H), 5.42 (s, 2H), 1.64-1.46 (m, 6H), 1.42-1.27 (m, 6H), 1.56-0.99 (m, 6H), 0.88 (t, J=7.3 Hz, 9H); ¹³C NMR (100.6 MHz, CDCl₃) δ 165.1, 163.5, 145.4, 142.0, 139.2, 136.7, 136.6, 136.0, 128.7, 128.4, 128.4, 128.3, 119.0, 115.8, 68.2, 67.2, 29.2, 27.5, 13.8, 9.7.

### Evaluation of Radioligands:

A series of lead] BChE-targeting radioligand candidates in three classes of molecules were synthesized, including pyridones: i) (p-iodophenyl)methyl 6-oxo-1H-pyridine-2-carboxylate (TRV7005), ii) (p-iodophenyl)methyl 1-methyl-6-oxo-1H-pyridine-2-carboxylate (TRV7006), iii) (p-iodophenyl)methyl 6-methoxy-2-pyridinecarboxylate (TRV7019), iv) benzyl 6-[(p-iodophenyl)methoxy]-2-pyridinecarboxylate TRV7040). The product profile of each radioligand was characterized based on their physicochemical attributes (using multiparametric optimization (MPO) scoring) and kinetic profile (evaluating standard enzyme kinetics parameters, including the maximum enzymatic reaction rate (Vₘₐₓ), rate of first chemical step (K_{cat}), Michaelis constant (Km), enzymatic efficiency (k_{cat}/Kₘ) and inhibition equilibrium constant (Kᵢ)). Radioligands were then imaged in vivo over one hour using two-dimensional (2D) dynamic planar SPECT scintigraphy in four strains of mice exhibiting differential expression of BChE. These mice included a familial AD mouse model (5XFAD) and corresponding wild-type (WT) counterparts in addition to BCHE-knockout BCHE-KO mice and a derived 5XFAD-BChE-KO strain both of which lack a BChE-expressing phenotype. SPECT imaging permitted determination of each radiotracer's ability to cross the blood-brain barrier (BBB) in addition to their biodistribution in the brain over time. Whole brain time-activity curves, generated from dynamic SPECT acquisitions binned into 60× 1-minute frames, were fit with a mono-exponential decay function from which the rate of tracer clearance, k_{clearance} (min₋₁), the half-life of tracer clearance, t_{1/2 clearance} (min) and asymptotic radiotracer concentration (i.e. tracer concentration remaining in the brain), C_{A} (% Cₘₐₓ) could be derived and evaluated. For each radioligand, these kinetic summary measures were then compared between the groups of mice imaged. Pooled data of mouse strains within each radioligand permitted an overall comparison of tracer kinetics between radioligands using an analysis of variance (ANOVA) statistical design.

### Radiosynthesis

[123I] radioligands were successfully synthesized with all radioligands achieving sufficiently good radiochemical yields using the methods below, with a range between 56.5-87% and high radiochemical purity, with a range between 95.2-99.9% among the radioligands evaluated.

### Example 9

### Synthesis of (p-[123I]Iodophenyl)methyl 6-oxo-1H-pyridine-2-carboxylate (TRV7005)

In a plastic microtube (250 μL), Na123I (˜185 MBq) in 0.1 M NaOH(aq) (20 μL) was diluted with 1 M NaOH(aq) (20 μL), then acidified with 1 M HCl(aq) (25.5 μL). The solution was vortexed, centrifuged and checked to ensure the pH was acidic. To this was added acetonitrile (50 μl) followed by (p-(tributylstannyl) phenyl)methyl 6-oxo-1H-pyridine-2-carboxylate in acetonitrile (50 μl, 4.64 mM). The reaction was initiated by adding N-chlorosuccinimide in acetonitrile (50 μl, 3 mM). After vortexing (7.5 minutes) at room temperature, the reaction mixture was centrifuged and injected in an Agilent Infinity 1260 HPLC with a 250 mm zorbax xdb eclipse C18 column with an eluent of 80% methanol and 20% water run at 1 ml per minute. After 6 minutes, the eluent was switched to 100% acetonitrile at 3 ml per minute to remove any unreacted precursor. Fractions were collected every 30 seconds. Using a retention time established with (p-Iodophenyl)methyl 6-oxo-1H-pyridine-2-carboxylate, the appropriate fractions containing the radioligand were collected and combined in a glass v-vial. The combined solution, with 50 μl of 1M HCl added, was dried at 55° C. under a light stream of argon. Radiotracer was re-dissolved in 5% ethanol and 0.9% saline (0.25 mL) for animal administration.

### Example 10

### (p-[123I]Iodophenyl)methyl 1-methyl-6-oxo-1H-pyridine-2-carboxylate (TRV7006)

In a plastic microtube (250 μL), Na123I (˜185 MBq) in 0.1 M NaOH(aq) (20 μL) was diluted with 1 M NaOH(aq) (15 μL), then acidified with 1 M HCl(aq) (20 μL). The solution was vortexed, centrifuged and checked to ensure the pH was acidic. The precursor, (p-(tributylstannyl)phenyl)methyl 1-methyl-6-oxo-1H-pyridine-2-carboxylate in acetonitrile (20 μl, 3 mM), was added and the reaction was initiated by adding N-chlorosuccinimide in acetonitrile (20 μl, 3 mM). After vortexing (7.5 minutes) at room temperature, the reaction mixture was centrifuged and injected in an Agilent Infinity 1260 HPLC with a 250 mm zorbax xdb eclipse C18 column with an eluent of 100% acetonitrile at 0.7 ml per minute. Fractions were collected every 30 seconds. Using a retention time established with (p-Iodophenyl)methyl 1-methyl-6-oxo-1H-pyridine-2-carboxylate, the appropriate fractions containing the radioligand were collected and combined in a glass v-vial. The combined solution, with 5 μl of 1 M HCl added, was dried at 55° C. under a light stream of argon. Radiotracer was re-dissolved in 5% ethanol and 0.9% saline (0.25 mL) for animal administration.

### Example 11

### (p-[123I]Iodophenyl)methyl 6-methoxy-2-pyridinecarboxylate (TRV7019)

Synthesis of (p-[123I]Iodophenyl)methyl 6-methoxy-2-pyridinecarboxylate was performed with modifications of a procedure described previously (DeBay, Reid, Pottie, et al., 2017). In a plastic microtube (250 μL), Na123I (˜185 MBq) in 0.1 M NaOH(aq) (20 μL) was diluted with 1 M NaOH(aq) (15 μL), then acidified with 1 M HCl(aq) (19.5 μL). The solution was vortexed, centrifuged and checked to ensure the pH was acidic. To this was added acetonitrile (50 μl) followed by (p-(tributylstannyl) phenyl)methyl 6-methoxy-2-pyridinecarboxylate in acetonitrile (50 μl, 3 mM). The reaction was initiated by adding N-chlorosuccinimide in acetonitrile (50 μl, 3 mM). After vortexing (7.5 minutes) at room temperature, the reaction mixture was centrifuged and injected in an Agilent Infinity 1260 HPLC with a 250 mm zorbax xdb eclipse C18 column with an eluent of 90% acetonitrile and 10% water run at 1 ml per minute. Fractions were collected every 30 seconds. Using a retention time established with (p-Iodophenyl)methyl 6-methoxy-2-pyridinecarboxylate, the appropriate fractions containing the radioligand were collected and combined in a glass v-vial. The combined solution, with 50 of 1M HCl added, was dried at 55° C. under a light stream of argon. Radiotracer was re-dissolved in 5% ethanol and 0.9% saline (0.25 mL) for animal administration.

### Example 12

### benzyl 6-[(p-[123I]iodophenyl)methoxy]-2-pyridinecarboxylate (TRV7040)

In a plastic microtube (250 μL), Na123I (˜185 MBq) in 0.1 M NaOH(aq) (20 μL) was diluted with 1 M NaOH(aq) (10 μL), then acidified with 1 M HCl(aq) (17 μL). The solution was vortexed, centrifuged and checked to ensure the pH was acidic. To this was added acetonitrile (50 μl) followed by benzyl 6-[(p-(tributylstannyl) phenyl)methoxy]-2-pyridinecarboxylate in acetonitrile (25 μl, 3 mM). The reaction was initiated by adding N-chlorosuccinimide in acetonitrile (25 μl, 3 mM). After vortexing (7.5 minutes) at room temperature, the reaction mixture was centrifuged and injected in an Agilent Infinity 1260 HPLC with a 250 mm zorbax xdb eclipse C18 column with an eluent of 90% acetonitrile and 10% water run at 1 ml per minute. Fractions were collected every 30 seconds. Using a retention time established with Benzyl 6-[(p-iodophenyl)methoxy]-2-pyridinecarboxylate, the appropriate fractions containing the radioligand were collected and combined in a glass v-vial. The combined solution was dried at 55° C. under a light stream of argon. Radiotracer was re-dissolved in 5% ethanol and 0.9% saline (0.25 mL) for animal administration.

### Physicochemical Profile of [¹²³I] Radioligands

After synthesis, multiparametric optimization (MPO) scores were determined as outlined by Wager et al. (Wager et al., 2010). Six physicochemical properties of each radioligand including molecular weight (MW), topological polar surface area (TPSA) most basic center (pKₐ), calculated partition coefficient (clogP), calculated distribution coefficient at pH 7.4 (clogD) and hydrogen bond donors (HBD) were determined. These properties were evenly weighted and scored between 0-1 to give a composite MPO score. Typically, an MPO ≥4.0 is predictive of a radioligand's ability to cross the blood-brain barrier (BBB) and reach the brain.

In general, the radioligands possessed favourable physicochemical characteristics. A summary of all six physicochemical properties that comprise the MPO score for a given radioligand is seen in Table 1.1. A central nervous system (CNS) radioligand with MPO ≥4 has a high probability of crossing the blood brain barrier. A desirable CNS radioligand typically possesses a lower MW (≤360 g/mol), possess a TPSA between 40-90, have fewer HBD (≤0.5), and favours smaller ClogP (≤3), ClogD (≤2) and pKa (≤8) values.

TABLE 1.1
Physicochemical profile of lead butyrylcholinesterase (BChE) [¹²³I] radioligands.

| | | | MW | | | | | |
|---|---|---|---|---|---|---|---|---|
| TRV # | Structure | Name | (g/mol) | TPSA | pKa | clogP/clogD | HBD | MPO |
| TRV7005 | 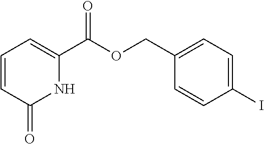 [SMILES: O=C(OCc1ccc(I)cc1)c1cccc(=O)[nH]1] | (p-Iodophenyl)methyl 6-oxo-1H-pyridine-2- carboxylate | 355.13 | 55.4 | 9.51 | 2.68/2.67 | 1 | 4.74 |
| TRV7006 | 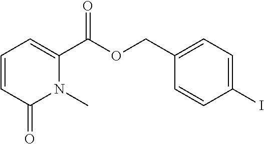 [SMILES: Cn1c(C(=O)OCc2ccc(I)cc2)cccc1=O] | (p-Iodophenyl)methyl 1-methyl-6-oxo-1H- pyridine-2- carboxylate | 369.15 | 48.33 | 20 | 2.9/2.9 | 0 | 4.50 |
| TRV7019 | 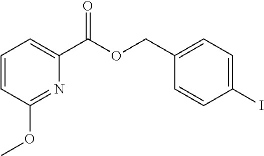 [SMILES: COc1cccc(C(=O)OCc2ccc(I)cc2)n1] | (p-Iodophenyl)methyl 6-methoxy-2- pyridinecarboxylate | 369.15 | 48.42 | 20 | 4.04/4.04 | 0 | 3.43 |
| TRV7040 | 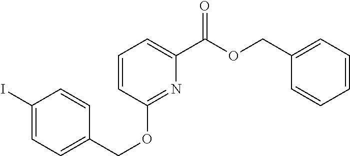 [SMILES: O=C(OCc1ccccc1)c1cccc(OCc2ccc(I)cc2)n1] | Benzyl 6-[(p- iodophenyl)methoxy]- 2-pyridinecarboxylate | 445.26 | 48.42 | 20 | 5.76/5.76 | 0 | 2.41 |

### In Vitro Enzyme Kinetics

In vitro enzyme kinetics were carried out using standard methods that have been described previously (Darvesh, Walsh, et al., 2003). Enzyme kinetic parameters included the maximum enzymatic reaction rate (Vₘₐₓ (M⋅min⁻¹)), rate of first chemical step (K_{cat} (min⁻¹)), Michaelis constant (Kₘ), enzymatic efficiency (k_{cat}/Kₘ) and inhibition equilibrium constant (Kᵢ (min⁻¹)).

Evaluation of enzyme kinetic revealed that [¹²³I] radioligands in the current study had favourable in vitro kinetic profiles, suggesting suitable specificity for BChE. In general, an effective BChE radioligand possesses a higher value of Vₘₐₓ, k_{cat}, k_{cat}/Kₘ and Kᵢ and a lower Kₘ value. The radiosynthesis of these tracers resulted in a radiochemical yield (RCY) between (56.5-87)% and a radiochemical purity (RCP) between (95.2-99.9)%. A complete summary of in vitro enzyme kinetic parameters is seen in Table 1.2.

TABLE 1.2
In vitro enzyme kinetic parameters and radiosynthesis results of candidate butyrylcholinesterase

| | | | | | | | | Radio- | |
|---|---|---|---|---|---|---|---|---|---|
| | | | Enzyme Kinetics | | | | | synthesis | |
| | | | Vₘₐₓ | k_{cat} | | K_{cat}/ | Kᵢ | RCY | RCP |
| TRV # | Structure | Name | (M•min⁻¹) | (min⁻¹) | Kₘ | Kₘ | (min⁻¹) | (%) | (%) |
| TRV7005 | 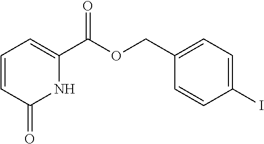 [SMILES: O=C(OCc1ccc(I)cc1)c1cccc(=O)[nH]1] | (p- Iodophenyl) methyl 1-methyl- 6-oxo-1H- pyridine-2- carboxylate | | 4900 | 1.5E-5 | 3.3 × 10⁸ | 4.38 × 10⁻⁵ | 68 | 96.3 |
| TRV7006 | 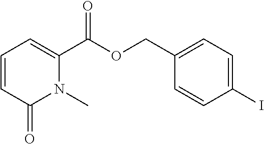 [SMILES: Cn1c(C(=O)OCc2ccc(I)cc2)cccc1=O] | (p- Iodophenyl) methyl 6- methoxy-2- pyridine- carboxylate | | 3000 | 2.1E-5 | 1.5 × 10⁸ | 3.1 × 10⁻⁵ | 56.5 | 95.2 |
| TRV7019 | 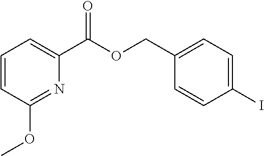 [SMILES: COc1cccc(C(=O)OCc2ccc(I)cc2)n1] | Benzyl 6-[(p- iodophenyl) methoxy]- 2-pyridine- carboxylate | | 80000 | 1.1 × 10⁻⁴ | 7.6 × 10⁸ | 8.8 × 10⁻⁶ | 74.2 | 99.9 |
| TRV7040 | 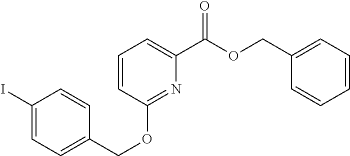 [SMILES: O=C(OCc1ccccc1)c1cccc(OCc2ccc(I)cc2)n1] | p- Iodophenyl- amino benzoate | | n/a | n/a | n/a | | 78.7 | 98.8 |

### 2D Dynamic Planar Single Photon Emission Computed Tomography (SPECT) Scintigraphy

### Example 13

Six BChE radioligand were administered through IV tail vein injections into mice 2 minutes after the start of a 60-minute SPECT scintigraphy scan that was acquired in the sagittal plane. At least two hours prior to imaging, mice were weighed and given an intraperitoneal (IP) injection of Lugol's solution (potassium iodide (KI)), dosed at 8.63 uL/g to block potential accumulation of [¹²³I]-labelled radiotracer in the thyroid, a gland that avidly uptakes iodinated molecules. Mice were then placed in an induction chamber, anaesthetized with 3% isofluorane (in 97% oxygen) and restrained in a TailVeiner Restrainer (Braintree Scientific Inc., Braintree MA, USA) while under a continuous stream of 1.5% isofluorane gas. A custom built, in-house catheter line (30-gauge, 0.5 inch needle; 0.025/0.012 inch polyethylene tubing, Braintree Scientific Inc., Braintree MA, USA) was placed in the lateral tail vein. Mice were then secured in prone position, wrapped in a blanket on a heated animal bed and maintained under continuous stream of 1.5-2% isofluorane while the respiration rate monitored for the duration of the imaging procedure (SA Instruments Inc. Stony Brook, NY). Scans were acquired with a SPARKTM SRT-50 tabletop SPECT scanner (Cubresa Inc., MB, CA) equipped with a 1 mm diameter single pinhole tungsten collimator (SciVis) with a transaxial aperture field of view (FOV) of 30 mm and inherent sensitivity of 50 cps/MBq. The mouse head region was centered on the scanner's FOV and demarked with fiducial markers incubated with [¹²³I] and integrated within the imaging bed. A 2D planar scintigraphy acquisition was initiated, acquired as a continuous sagittal projection over 60 minutes. Two minutes after the start of the scan, each radioligand was administered through the tail vein catheter line over ˜15 sec and subsequently flushed with ˜20 μL saline. (A summary of the average injected doses of each radioligand is seen in Table 1.4 below.) Whole brain time-activity curves were generated from which a single exponential function was fitted to determine kinetic parameters of tracer clearance from the brain. Goodness of fit metrics for each radioligand are seen in Table 1.3 and in general, indicate a strong correspondence of the fitted curves with the measured SPECT time-activity curve data, suggesting that this approach adequately describes the clearance characteristics for each radiotracer. R², Sum of squares (SS), and standard deviation of residuals (Sy.x) were evaluated for the brain clearance curves for each radioligand. TRV 7040 was not evaluated as it did not cross the blood-brain barrier.

TABLE 1.3
Goodness of fit metrics for each [¹²³I] radioligand modeled by a single decreasing
exponential function. Mean ± SEM shown in addition to the range of values in parentheses.
Smaller SS, Sy.x values and R² values closer to 1.0 indicating better overall fits.

| | Goodness of fit | | |
|---|---|---|---|
| | mean ± SEM (range) | | |
| [¹²³I] Radioligand | R² | SS | Sy.x |
| TRV7005 | 0.93 ± 0.02 (0.76-0.98) | 446 ± 59 (122-684) | 2.87 ± 0.17 (2.05-3.56) |
| TRV7006 | 0.92 ± 0.02 (0.88-0.97) | 392 ± 52 (271-494) | 2.68 ± 0.18 (2.24-3.03) |
| TRV7019 | 0.95 ± 0.01 (0.93-0.98) | 159 ± 13 (132-202) | 1.71 ± 0.07 (1.57-1.93) |
| TRV7040 | | | |

2D SPECT scintigraphy projections were converted to list mode data using built-in Cubresa SPARKTM preprocessing routine at 159 keV with a 20% energy window applied. Images were reconstructed to a 208×208 matrix, yielding a resolution of 0.8 mm. List mode data of the 60 minute scan were then re-binned into 60 second frames giving an effective temporal resolution of 60 sec for each projection image.

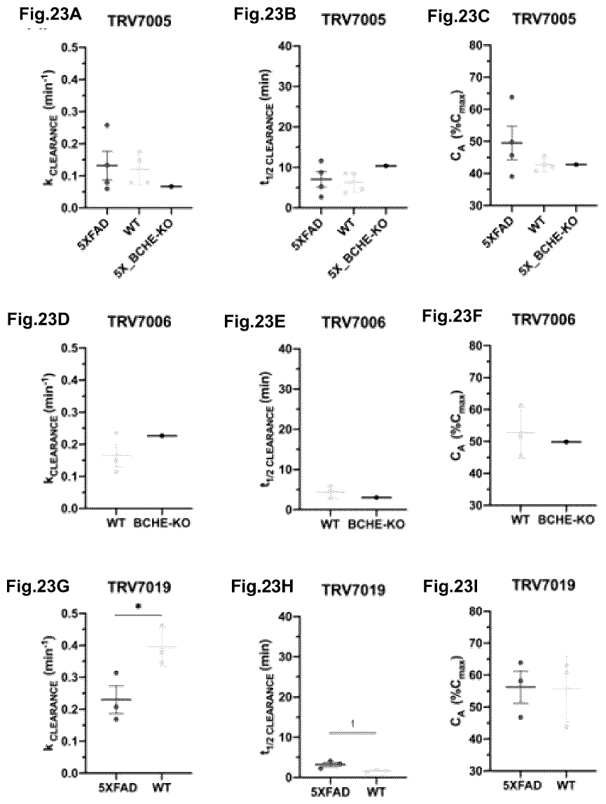

In general, SPECT scintigraphy projections of the mouse brain showed sufficient image quality, generating between (3.20±0.75)−(9.21±1.38) million counts across radioligands (Table 1.4). SPECT images could delineate whole brain from other extracranial structures in close proximity. Differential patterns of distribution were observed over time between the various radioligands evaluated, as detailed in subsequent sections. In the subsequent sections, time-activity curves for each radioligand are shown in sequence following the SPECT scintigraphy image series and are also shown in a combined summary panel at the end of the current section (FIG. 23).

TABLE 1.4
2D dynamic planar SPECT scintigraphy subjects demographics, grouped by butyrycholinesterase (BChE) [¹²³I] radioligand.

| | | | | | | | | ID |
|---|---|---|---|---|---|---|---|---|
| | | | | | | | | (mCi) |
| | | | | | | BCHE- | | Total |
| | | IUPAC | | 5XFADₙ | WTₙ | KOₙ | Age | Cts |
| TRV # | Structure | Name | Modality | (M,F) | (M,F) | (M,F) | (months) | (×10⁶) |
| TRV7005 | 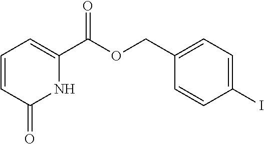 [SMILES: O=C(OCc1ccc(I)cc1)c1cccc(=O)[nH]1] | (p- Iodophenyl) methyl 6-oxo-1H- pyridine- 2- carboxylate | ¹²³I SPECT 2D dynamic planar scinti- graphy | 4 (4M,0F) | 4 (4M,0F) | 1 (5X- BCHE- KO) (1M,0F) | (11.2 ± 0.2) | (1.45 ± 0.114) mCi (9.08 ± 1.12) |
| TRV7006 | 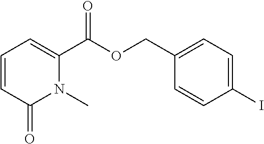 [SMILES: Cn1c(C(=O)OCc2ccc(I)cc2)cccc1=O] | (p- Iodophenyl) methyl 1-methyl- 6-oxo- 1H- pyridine-2- carboxylate | ¹²³I SPECT 2D dynamic planar scinti- graphy | n/a | 3 (0M,3F) | 1 (BCHE- KO) (0M,1F) | (12.3 ± 0.0) | (0.959 ± 0.029) mCi (6.56 ± 0.15) |
| TRV7019 | 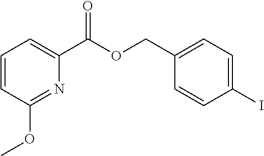 [SMILES: COc1cccc(C(=O)OCc2ccc(I)cc2)n1] | (p- Iodophenyl) methyl 6-methoxy- 2- pyridine- carboxylate | ¹²³I SPECT 2D dynamic planar scinti- graphy | 3 (0M,3F) | 3 (OM,3F) | n/a | (12.7 ± 0.3) | (1.398 ± 0.200) mCi (9.21 ± 1.38) |
| TRV7040 | 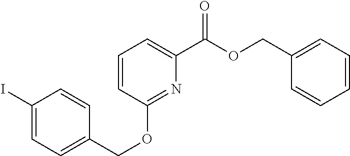 [SMILES: O=C(OCc1ccccc1)c1cccc(OCc2ccc(I)cc2)n1] | Benzyl 6- [(p- iodophenyl) methoxy]-2- pyridine- carboxylate | ¹²³I SPECT 2D dynamic planar scinti- graphy/ 3D SPECT | 2 (2M,0F) | 2 (1M,1F) | n/a | (8.4 ± 1.1) | (1.175 ± 0.0130) mCi (7.15 ± 0.86) |

IUPAC = International Unit of Pure and Applied Chemistry nomenclature; n = number of subjects; M = male; F = female; ID = injected dose in
millicuries (mCi); Total cts = total counts of radioactive decay acquired over scan.
Mean ± SEM.

### 2D Planar SPECT Scintigraphy Imaging/Statistical Analyses

At the frame of peak tracer concentration in the brain (Cmax), a 5 mm×15 mm rectangular ROI was manually placed over the whole brain projection (aligned with the fiducial markers) and then propagated over each of the remaining 59 frames for analysis in VivoQuant® (Invicro, Boston, MA). Sagittal projections were acquired dynamically over 60 minutes with radioligand administration occurring 2 minutes after the start of the scan. Images were binned into 60× 1-minute frames for analysis (frames 1-40 displayed in the figures, denoted numerically in white at the top left of each image). A fused CT/MRI overlay serves as an anatomical reference in frame 1 indicating brain (yellow arrow), heart (red arrow), thyroid/salivary gland region (green arrow) and integrated fiducial marker placed at the most rostral extent of the olfactory bulb (purple arrow). D=dorsal; V=ventral, R=rostral; C=caudal. Time-activity curves were expressed as a percentage relative to the peak concentration (i.e. % Cₘₐₓ) of tracer that reached the brain.

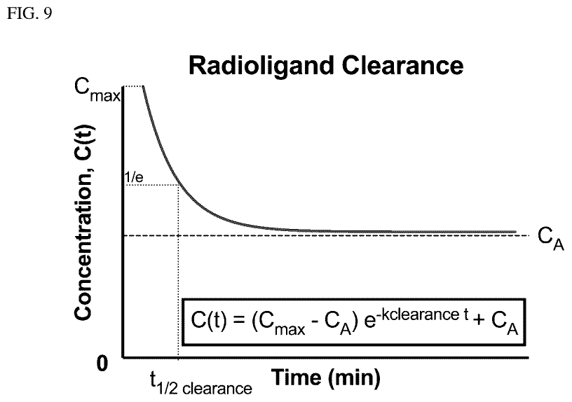

Radioligand concentration in the brain as a function of time, C(t) is shown in FIG. 9, which depicts the single-phase clearance of a radiotracer from the brain with an initial peak concentration, Cₘₐₓ. Clearance is expressed as a decreasing monoexponential function with associated kinetic parameters t_{1/2 clearance} (the half-life of tracer clearance (min)), k_{clearance} (the rate of tracer clearance (min⁻¹)), and CA (the asymptotic tracer concentration, expressed as % Cₘₐₓ). The brain retention characteristics of each radioligand were evaluated by fitting a single mono-exponential decay function, via iterative least squares method to the respective radioligand's time-activity curves depicting % Cₘₐₓ in the brain. Exponential fits were constrained to start at a peak brain concentration, Cₘₐₓ, of 100%. Goodness of fit of the resultant exponential curves was evaluated using R² and standard deviation of the residuals (Sy,x) metrics, with higher R² and lower Sy.x values indicating fits that better-describe the overall data. Exponential fitting was performed in PRISM 8.0 (GraphPad, San Diego, CA). Three derived parameters of the exponential fits were evaluated, providing summary measures of radiotracer clearance, namely, the rate constant of tracer clearance, k_{clearance} (min⁻¹), the half-life of tracer clearance, t_{1/2 clearance} (min), and the asymptotic concentration of tracer, C_{A} (% Cₘₐₓ).

For each radioligand, group means of these derived summary measures were compared between mouse strains imaged using independent samples student T-tests at a significance level of 5% (p<0.05), assuming equal variances (to permit statistical comparison among even those groups with only single mice represented).

### TRV7005

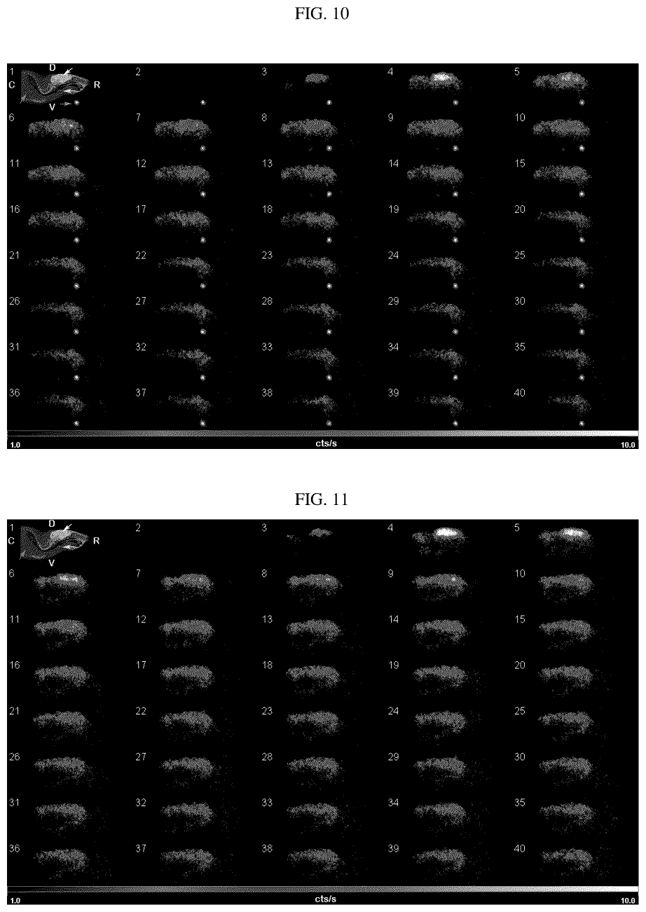

2D dynamic planar SPECT scintigraphy scans revealed significant whole brain uptake of TRV7005, confirming the ability of TRV7005 to cross the blood-brain barrier (BBB). Representative images series for 5XFAD and WT mice are seen in FIG. 10 and FIG. 11, respectively. FIG. 10 shows dynamic planar SPECT scintigraphy scans for a representative 5XFAD mouse and FIG. 11 shows dynamic planar SPECT scintigraphy scans for a representative WT mouse.

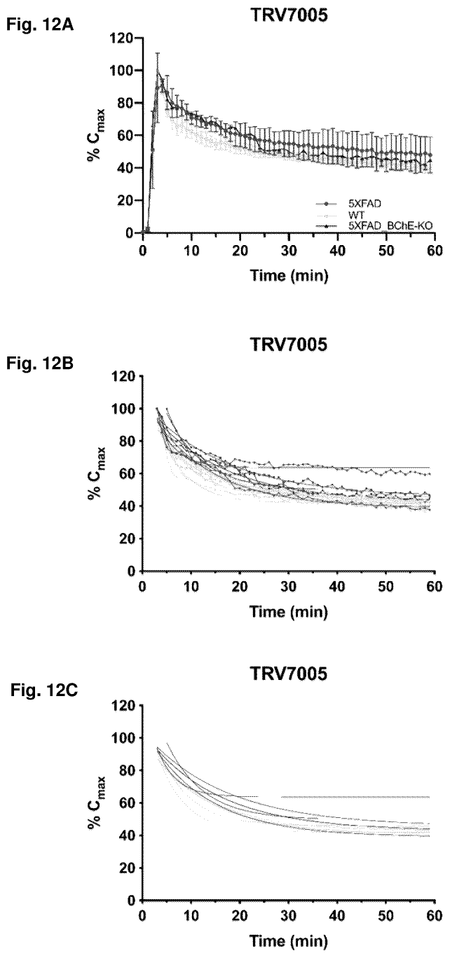

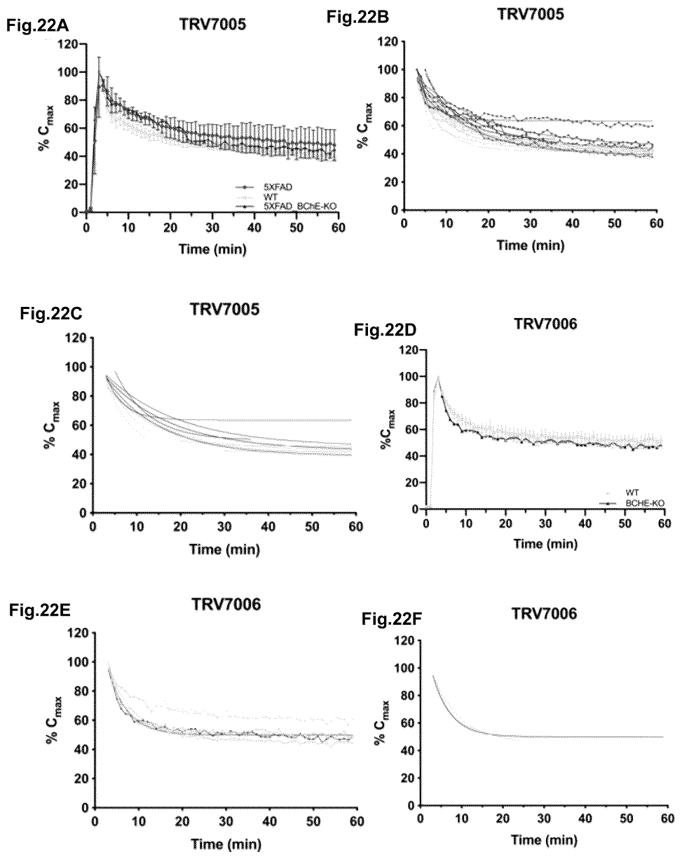

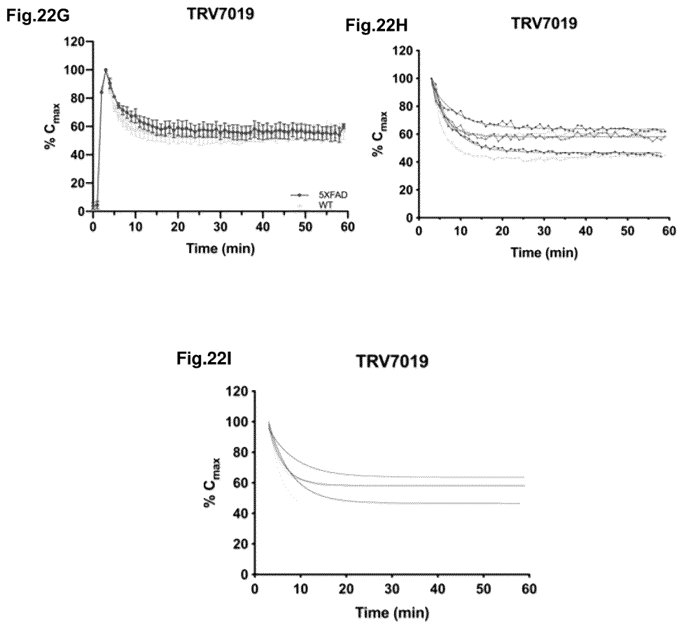

Peak radioligand uptake occurred in the 5XFAD and WT brain at frame 4 (2 minutes post-injection) in all but one WT mouse (frame 5), with uptake largely restricted to the brain and subsequent washout of TRV7005 from the brain in both 5XFAD and WT mice was apparent by visual inspection over the scan duration. (FIGS. 10 and 11) In both the 5XFAD and WT mice, radiotracer uptake was largely restricted to the brain with subsequent washout of TRV7005 apparent over the course of the scan. A diffusely distributed background signal was also seen in extracranial regions within the animal as the scan progressed. Corresponding whole brain time-activity curves are seen in FIG. 12, FIG. 22 and associated kinetic summary measures of tracer clearance are seen in FIG. 23, Table 1.5. Colour scale in the Figures represent average counts per second (cts/s) in a given image pixel.

The whole brain time-activity curves (expressed as percent of peak concentration (% Cₘₐₓ) in brain) seen in FIG. 12 are presented as: A) mean time activity curves (mean±SEM) are shown for WT (green), 5XFAD (blue) and 5XFAD-BChE-KO (black) (FIG. 12A, FIG. 22A); B) time-activity curves having individual mice subject data and corresponding fitted exponential function (solid line) (FIG. 12B, FIG. 22B) and fitted exponential functions alone for individual mice from which kinetic summary measures were derived and compared (FIG. 12C, FIG. 22C). Mean time-activity curves for 5XFAD and WT were similar, with considerable overlap apparent between the time-activity curves of the two groups (FIG. 12A), save a subtle divergence of the curves observed between 5-15 minutes suggesting a short period of latent clearance in the 5XFAD brain, that subsequently reaches values similar to that of WT. Nevertheless, no significant differences were observed in the overall rate of tracer clearance, k_{clearance} (5XFAD=0.12±0.03 min⁻¹; WT=0.12±0.02 min⁻¹; p=0.971) the half-life of clearance, t_{1/2clearance} (5XFAD=6.51±1.19 min; WT=7.21±1.75 min; p=0.756) or asymptotic tracer concentration, C_{A} (5XFAD=49.2±5.10%; WT=42.5±1.03%; p=0.281) between 5XFAD and WT groups (FIG. 23A-C, Table 1.5).

### TRV7006

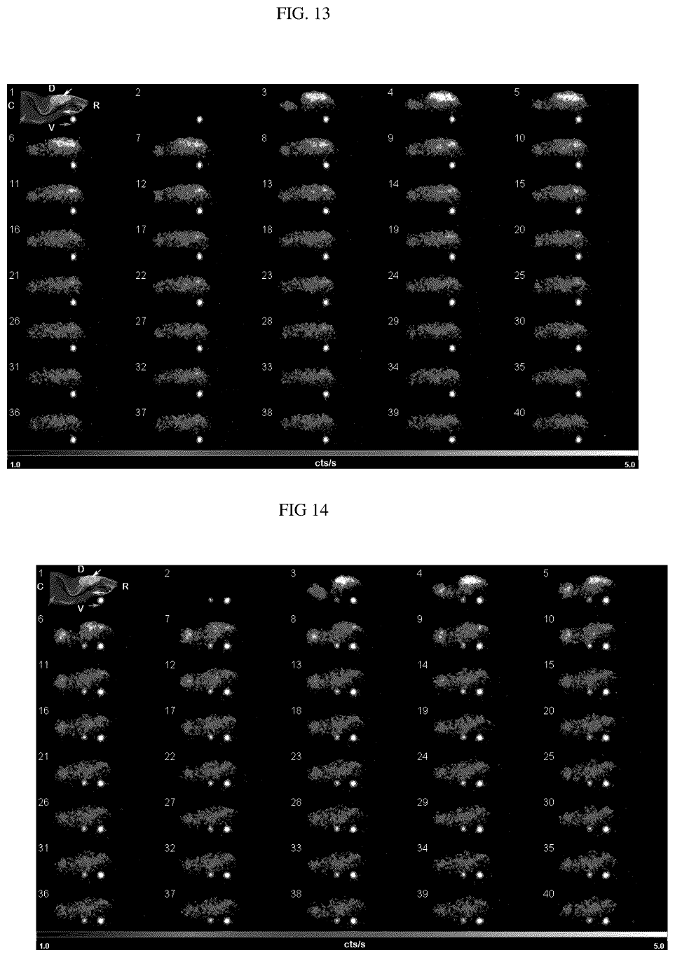

TRV7006, a structurally similar radioligand to TRV7005, also readily crosses the BBB and is taken up in the brain. Representative 2D dynamic planar SPECT scintigraphy scans for WT and BChE-KO mice are seen in FIG. 13 and FIG. 14, respectively.

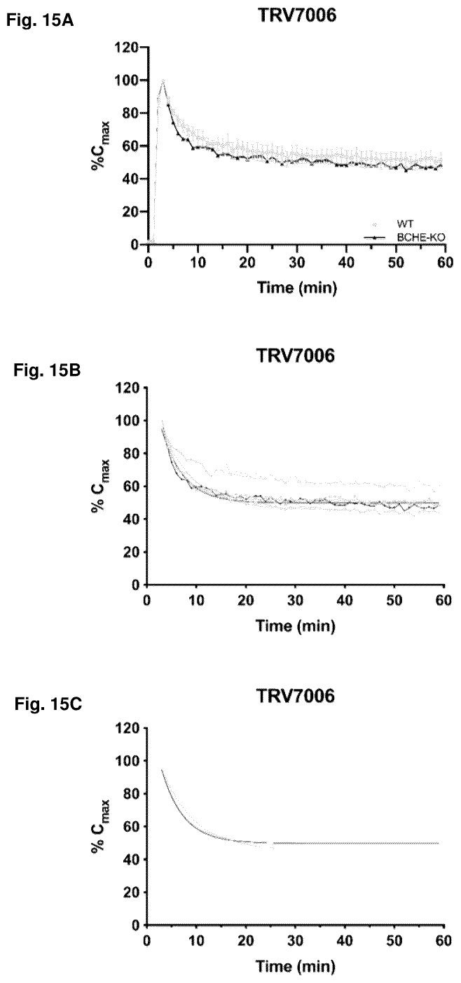

Peak radioligand uptake reliably occurred at frame 4 (2 minutes post-injection) in the WT and BChE-KO mice. A similar pattern of brain uptake was observed to that of TRV7005 (FIG. 13, FIG. 14). Washout of TRV7006 also occurred in both WT and BChE-KO mice. A diffuse distribution of background signal is also seen extracranially within the animal over the course of the scan with additional cardiac signal accumulation at early time points for the WT mice and with prominent cardiac signal accumulation at early time points for the BCheE-KO mice. Corresponding whole brain time-activity curves are seen in FIG. 15, FIG. 22 and associated kinetic summary measures of tracer clearance are seen in FIG. 23, Table 1.5. Colour scale represents average counts per second (cts/s) in a given image pixel.

The whole brain time-activity curves (expressed as percent of peak concentration (% Cₘₐₓ) seen in FIG. 15 are presented as: A) mean time-activity curves (mean±SEM) are shown for WT (green) and BChE-KO (black); B) time-activity curves having individual mice subject data and corresponding fitted exponential function (solid line); and C) fitted exponential functions alone for individual mice from which kinetic summary measures were derived and compared (Table 1.5, FIG. 23). Mean time-activity curves for WT and BChE-KO demonstrated considerable overlap apparent between the time-activity curves of the two groups (FIG. 1.7A). No significant differences were observed in the rate of clearance k_{clearance} (WT=0.16±0.06 min⁻¹; BChE-KO=0.21±0.03 min⁻¹; p=0.525) t_{1/2clearance} (WT=4.72±0.93 min; BChE-KO=3.27±0.93 min; p=0.517) or asymptotic tracer concentration, CA (WT=52.3±4.6%; BChE-KO=49.3±4.6, p=0.770) between WT and BChE-KO mice (FIG. 23D-F, Table 1.5).

### TRV7019

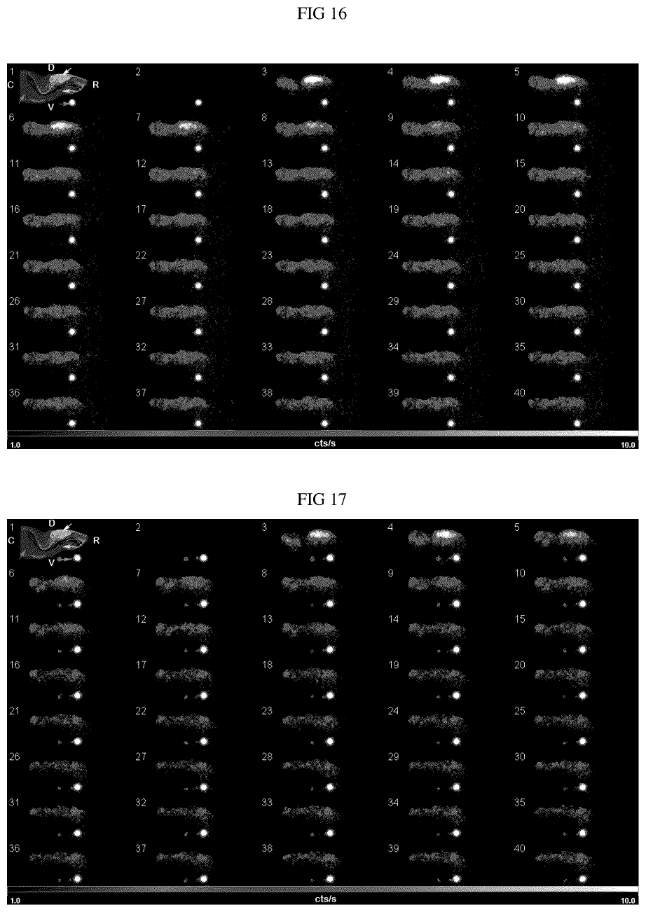

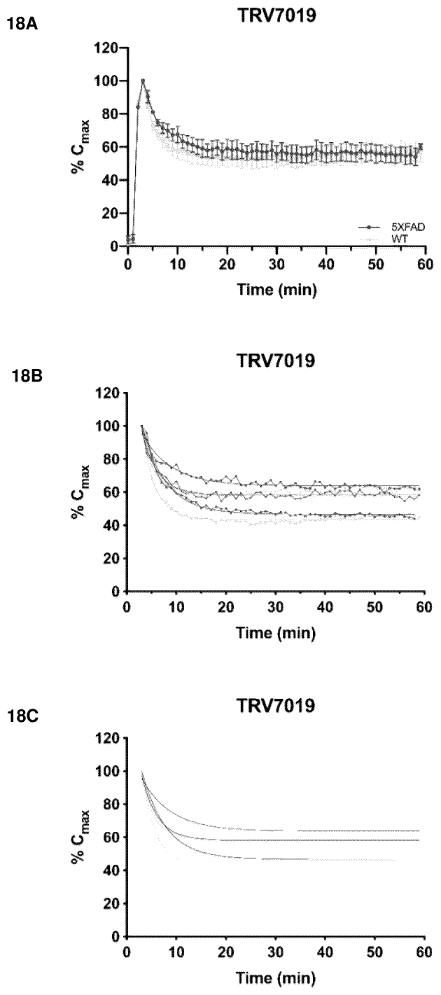

TRV7019 also readily crosses the BBB and is taken up in the brain. Representative 2D dynamic planar SPECT scintigraphy scans for 5XFAD and WT mice are seen in FIG. 16 and FIG. 17, respectively.

Peak brain perfusion reliably occurred at frame 3 (1-minute post-injection) in the 5XFAD and WT mice investigated, with prominent tracer uptake in the brain and notably, unlike TRV7005 and TRV7006, sustained cardiac tracer signal accumulation was a notable feature (FIG. 16 and FIG. 17) in addition to diffusely distributed background signal throughout the body. Washout of TRV7019 also occurred in both 5XFAD and WT mice. Corresponding whole brain time-activity curves are seen in FIG. 18, FIG. 22 and associated kinetic summary measures of tracer clearance are seen in FIG. 23, Table 1.5. Colour scale represents average counts per second (cts/s) in a given image pixel.

The whole brain time-activity curves (expressed as percent of peak concentration (% Cₘₐₓ) in brain) seen in FIG. 18 are presented as: A) mean time-activity curves (mean±SEM) are shown for WT (green) and 5XFAD (blue); B) time-activity curves of individual mice and corresponding fitted exponential function (solid line); and C) fitted exponential functions alone for individual mice from which kinetic summary measures were derived and compared (Table 1.5, FIG. 23). Mean time-activity curves for 5XFAD and WT (FIG. 18A), showed a divergence of the curves observed between 5-15 minutes, similar to what was observed with TRV7005. Significant differences were observed in kinetic summary measures between 5XFAD and WT, where a 58% decrease in k_{clearance} (5XFAD=0.23±0.4 min⁻¹; WT=0.4±0.04 min⁻¹; p=0.041) (FIG. 23G) and commensurate increase in t_{1/2clearance} (SXFAD=3.22±0.55 min; WT=1.77±0.15 min p=0.065 (statistical trend)) (FIG. 23H) were demonstrated in the 5XFAD brain. However, no significant difference in asymptotic tracer concentration, C_{A}(5XFAD=56.2±5.0%; WT=55.9±6.1%, p=0.964) was apparent between 5XFAD and WT groups (FIG. 23I). A summary of these tracer clearance metrics is seen in FIG. 23G-I and Table 1.5.

### TRV7040

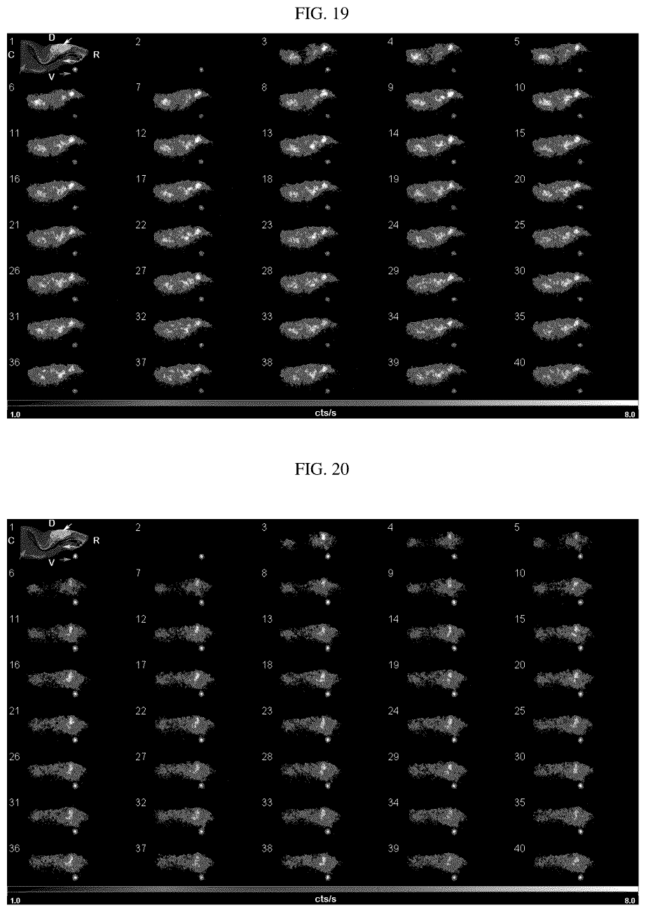

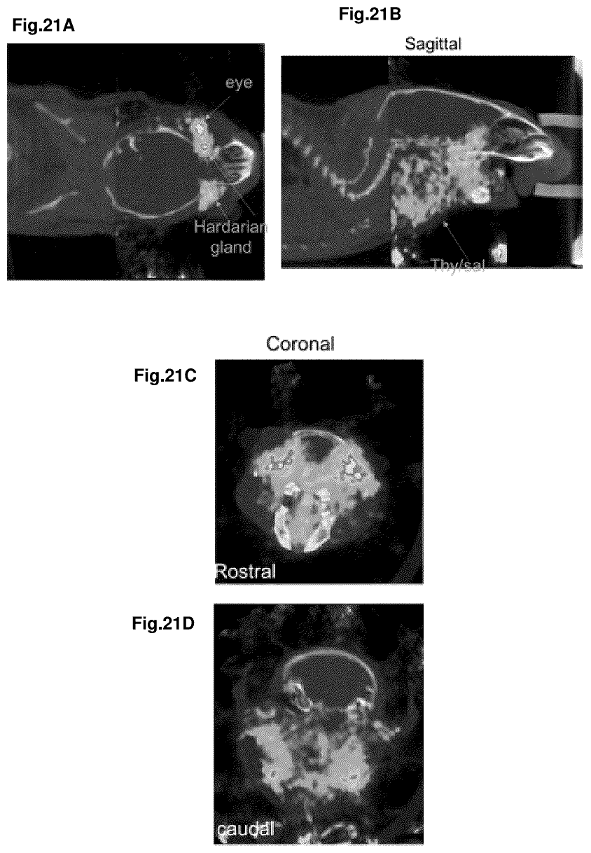

SPECT planar imaging of TRV7040 revealed an apparent inability of the radioligand to cross the BBB in appreciable amounts. Representative images series of TRV7040 for 5XFAD and WT mice are seen in FIG. 19 and FIG. 20, respectively. Within 1 minute after TRV7040 injection (frame 3), signal accumulation is apparent in the heart (with strong cardiac signal accumulation a feature of the scan of FIG. 19 for 5XFAD mice) and avid uptake of tracer in the ocular region and proximal arteries that serve this area is observed. Sustained retention in this region occurs over the duration of the 1-hour scan. Tracer accumulation in thyroid and salivary gland regions gradually emerges by 8 minutes post-injection (Frame 10), and is maintained over the duration of the scan.

Where no clear uptake in the brain was apparent, further tracer kinetic analyses were not carried out. However, 3D SPECT imaging was investigated to evaluate the precise distribution of the tracer in vivo. A 5XFAD mouse received an IV injection of of TRV7040 (1.27 mCi in 210 uL), followed by a saline flush of ˜10 μL. Tracer uptake occurred in conscious mice over 10 minutes. The mouse head region was centered on a 14 mm axial field of view (FOV) and a 3D static SPECT scan was acquired in super list mode (SLM) over 40 minutes (4 projections) on the SPARKTM SRT-50 single head standalone tabletop SPECT scanner (Cubresa Inc., Winnipeg, MB) integrated with a Triumph XO LabPET pre-clinical computed tomography (CT) scanner (Trifoil Imaging, CA). Following SPECT imaging, a CT scan was acquired for anatomical reference. CT images were collected in fly mode with a 70 kVp x-ray beam energy (160 μA beam current), 512 projections, 4 summed frames/projection, with 2×2 binning and magnification of 2.26×, providing complete whole brain coverage in a 56 mm FOV. CT scan duration was 8.5 min.

Results confirm the 2D scintigraphy findings, with negligible radioligand reaching the brain. However, avid uptake of TRV7040 is seen in the eye and hardarian glands with a focal distribution of radiotracer seen in these regions (FIG. 21). This is consistent with the 2D dynamic planar scintigraphy scans acquired (FIG. 19 and FIG. 20).

### Comparison of Kinetics Between Radioligands

To compare the clearance behaviour between radioligands, data were pooled across each radiotracer (combining available 5XFAD, WT, BChE-KO and 5XFAD-BChE-KO mice) to generate mean time-activity curves. In the ANOVA statistical design, if significant interaction was present (p<0.05), post-hoc comparisons were made using least significant difference (LSD) comparisons.

Overall mean time-activity curves for each radioligand are shown in FIG. 22A Differing clearance characteristics are apparent by qualitative inspection of these curves, suggesting differential brain retention among the radioligands evaluated. Pooled kinetic summary measures between radioligands were compared using ANOVA. Main effects of tracer clearance rate, k_{clearance} (p=0.0001), half-life of clearance, t_{1/2clearance} (p=0.0015), and asymptotic tracer concentration C_{A} (p=0.0011) were significant.

FIG. 22 shows whole brain time-activity curves for TRV7005 (A,B,C), TRV7006 (D,E,F), TRV7019 (G,H,I) radiotracers, expressed as % of peak concentration (% Cₘₐₓ) in brain. Mean time-activity curves (mean±SEM) are shown in the left column across the strains evaluated for each respective radioligand (blue=5XFAD; green=WT; black=BChE-KO or 5XFAD-BChE-KO). Time-activity curves of individual mice within a strain are shown in the middle column with corresponding exponential fit (solid line). Exponential fits alone are seen in the right column for each radioligand, from which kinetic summary measures were derived and compared (FIG. 23, Table 1.5).

FIG. 23 shows kinetic summary measures of tracer clearance from the brain for TRV7005 (A,B,C), TRV7006 (D,E,F), TRV7019 (G,H,I) radioligands. For each radioligand, the rate of tracer clearance, k_{clearance} (min⁻¹), shown in the left column, half-life of tracer clearance t_{1/2clearance} (min), shown in center column, and asymptotic tracer concentration, CA (% Cₘₐₓ), shown in right column were compared between 5XFAD (blue), WT (green) and in some instances BChE-KO (black) and 5XFAD-BChE-KO (black) mice. Mean (±SEM). No significant differences between mouse strains were observed in k_{clearance}, t_{1/2clearance} or CA for TRV7005, TRV7006 (p≥0.281). A 58% decrease in k_{clearance} (5XFAD=0.23±0.04; WT=0.40±0.04; p=0.041) and a commensurate increase in t_{1/2clearance} (5XFAD=3.22±0.55; WT=1.77±0.15; p=0.065 (statistical trend)) was observed with TRV7019; however, CA was not significantly different between 5XFAD and WT with TRV7019. *, p<0.05; †, p<0.10 (statistical trend).

Table 1.5 sets out single photon emission computed tomography (SPECT) dynamic planar scintigraphy kinetic summary measures of radioligand clearance for each of the radiotracers evaluated in 5XFAD, WT, BCHE-KO and 5XFAD-BCHE-KO mice (mean±SEM). The rate of tracer clearance (k_{clearance}), half-life of tracer clearance (t_{1/2clearance}) and asymptotic tracer concentration (CA) were compared (where possible) between mouse groups using an independent samples' t-test, assuming equal varainces. For TRV7019, k_{clearance} was significantly (58%) lower and a commensurate increase in t_{1/2clearance} in the 5XFAD brain was observed compared to WT; however, the final asymptotic radiotracer concentration in the brain, CA was not significantly different between 5XFAD and WT mice. No other significant differences were observed between mouse strains in each of the other radioligands evaluated. * denotes statistical significance (p<0.05) and † denotes a statistical trend (p<0.10). Entries with “n/a” indicate that no data was available and the greyed-out entry for TRV7040 are owed to the lack of blood-brain barrier penetrance required to evaluate brain radioligand kinetics.

TABLE 1.5
Single photon emission computed tomography (SPECT)
dynamic planar scintigraphy kinetic summary

| | k_{clearance} (min⁻¹) | | | | | |
|---|---|---|---|---|---|---|
| | | | BCHE-KO | | t_{1/2 clearance} (min) | |
| TRV# | 5XFAD | WT | 5XBCHE-KO | p value | 5XFAD | WT |
| TRV7005 | 0.12 ± 0.03 | 0.12 ± 0.02 | 0.07 | 0.971 | 6.51 ± 1.19 | 7.21 ± 7.75 |
| TRV7006 | n/a | 0.16 ± 0.03 | 0.21 ± 0.03 | 0.525 | n/a | 4.72 ± 0.93 |
| TRV7019 | 0.23 ± 0.04 | 0.40 ± 0.04 | n/a | 0.041* | 3.22 ± 0.55 | 1.77 ± 0..15 |
| TRV7040 | | | | | | |

| | t_{1/2 clearance} (min) | | C_{A} (% Cₘₐₓ) | | | |
|---|---|---|---|---|---|---|
| | BCHE-KO | | | | BCHE-KO | |
| TRV# | 5XBCHE-KO | p value | 5FXAD | WT | 5XBCHE-KO | p value |
| TRV7005 | 10.38 | 0.756 | 49.2 ± 5.1 | 42.5 ± 1.0 | 42.8 | 0.281 |
| TRV7006 | 3.27 ± 0.93 | 0.517 | n/a | 52.4 ± 4.6 | 49.3 ± 4.6 | 0.334 |
| TRV7019 | n/a | 0.65† | 56.2 ± 5.0 | 55.9 ± 6.1 | n/a | 0.964 |
| TRV7040 | | | | | | |

Table 1.6 sets out pooled in vivo kinetic summary measures for lead BChE radioligands. The rate of tracer clearance, k_{clearance} (min⁻¹), half-life of tracer clearance t_{1/2clearance} (min) and asymptotic tracer concentration, CA (% Cₘₐₓ) were compared using an ANOVA statistical design. TRV7040 was not taken up by the brain and thus not included for the current analysis.

TABLE 1.6
Pooled in vivo kinetic summary measures
for lead BChE radioligands.

| | in vivo kinetic Summary Metrics (pooled) | | | |
|---|---|---|---|---|
| TRV# | n | k_{clearance} (min⁻¹) | t_{1/2 clearance} (min) | C_{A} (% Cₘₐₓ) |
| TRV7005 | 9 | 0.11 ± 0.02 | 7.23 ± 0.95 | 45.4 ± 2.4 |
| TRV7006 | 4 | 0.17 ± 0.03 | 4.36 ± 0.75 | 51.6 ± 3.4 |
| TRV7019 | 6 | 0.31 ± 0.05 | 2.50 ± 0.41 | 56.1 ± 3.5 |
| TRV7040 | | | | |
| TRV5001 | 3 | 0.06 ± 0.02 | 17.4 ± 8.5 | 67.7 ± 5.3 |
| TRV6001 | 2 | 0.04 ± 0.01 | 18.8 ± 2.4 | 68.3 ± 1.2 |

### Results:

[¹²³I] Radioligands were successfully synthesized with all radioligands achieving sufficiently good radiochemical yields (56.5-87%) and radiochemical purity (95.2-95.9%). In general, most radioligands possessed favourable physicochemical characteristics. In vitro enzyme kinetics identified the radioligands as having high specificity for BChE. 2D dynamic planar SPECT scintigraphy provided sufficient image quality to evaluate the biodistribution of the radioligands evaluated. With the exception of TRV7040, all pyridone radioligands, including TRV7005, TRV7006 and TRV7019, crossed the BBB and were taken up in the brain. The MPO score served as a good predictor of BBB penetrance. The MPO of TRV7040 (MPO=2.41) was the only tracer significantly lower than the MPO threshold (≥4) and SPECT scintigraphy confirmed the inability of TRV7040 to cross the BBB and reach the brain. It is of note that TRV7040, is structurally similar to other radioligands in the same class of molecules, yet exhibits drastically different biodistribution behaviour in vivo.

Whole brain time-activity curves depicting radioligand concentration over time expressed relative to the peak concentration reached in the brain (% Cₘₐₓ) were generated from which a single exponential function was fitted to data allowing kinetic summary measures of tracer clearance to be successfully evaluated. Goodness of fit metrics, in general, indicate a strong correspondence of the fitted curves with the measured SPECT time-activity curve data suggesting that this approach adequately describes the clearance characteristics for each radiotracer. Exponential fits were also carried out on these data, producing near-identical results in estimates of k_{clearance} and t_{1/2clearance} to that obtained by time-activity curves expressed as % Cₘₐₓ. In general, no significant differences in k_{clearance}, t_{1/2clearance} and C_{A} were observed between mouse strains for each radioligand evaluated, save TRV7019, where a 58% decrease in k_{clearance} (5XFAD=0.23±0.4 min⁻¹; WT=0.4±0.04 min⁻¹; p=0.041) and commensurate increase of 82% in t_{1/2clearance} (5XFAD=3.22±0.55 min; WT=1.77±0.15 min p=0.065 (statistical trend)) were demonstrated. However, no significant difference in asymptotic tracer concentration, C_{A} (5XFAD=56.2±5.0%; WT=55.9±6.1%, p=0.964) was apparent between 5XFAD and WT groups.

In general, the BChE radioligands evaluated in the current study possessed favourable physicochemical characteristics, with all but one radioligand (TRV7040) crossing the blood brain barrier. 2D dynamic planar SPECT scintigraphy proved to be a robust technique with sufficient sensitivity to evaluate radioligand kinetics clearance and was able to distinguish different rates of clearance between radioligands in vivo. Even within the same class of radioligands, different clearance behaviour was apparent. For radioligand TRV7019, comparisons of 5XFAD and WT mice revealed significant differences in k_{clearance} and t_{1/2 clearance} between the two strains, with more rapid clearance of tracer in WT mice compared to 5XFAD, reaching similar overall concentrations at the end of the 60 minute scan. No other significant differences between mouse strains were detected in the radioligands evaluated. Interestingly, TRV7019 had the highest in vitro enzymatic efficiency (k_{cat}/kₘ) of the radioligands studied, yet clears the fastest.

### CONCLUSION

In the preceding specification, the invention has been described with reference to specific exemplary embodiments and examples thereof. It will, however, be evident that various modifications and changes may be made thereto without departing from the broader spirit and scope of the invention as set forth in the claims that follow. The specification and drawings are accordingly to be regarded in an illustrative manner rather than a restrictive sense.

1. A compound of Formula I: 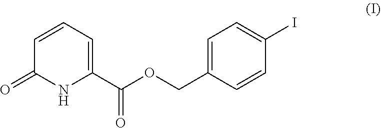 [SMILES: O=C(OCc1ccc(I)cc1)c1cccc(=O)[nH]1]
or a pharmaceutically acceptable salt thereof.

2. A compound of Formula II: 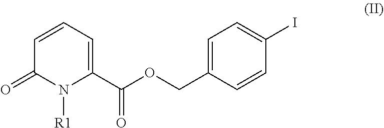
or a pharmaceutically acceptable salt thereof, in which R1 is alkyl.

3. A compound of Formula III: 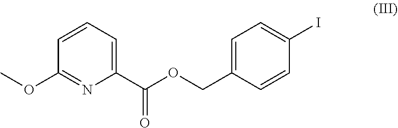 [SMILES: COc1cccc(C(=O)OCc2ccc(I)cc2)n1]
or a pharmaceutically acceptable salt thereof.

4. A method of diagnosis of an amyloid disease in a subject comprising administering a diagnostically effective amount of a compound of any of claims 1-3 to the subject.

5. The method of claim 4 in which the amyloid disease is Alzheimer's disease.

6. The method of claim 4 in which the amyloid disease is Parkinson's disease.

7. A method of diagnosis of multiple sclerosis in a subject comprising administering a diagnostically effective amount of a compound of any of claims 1-3 to the subject.

8. A method of diagnosis of brain tumour in a subject comprising administering a diagnostically effective amount of a compound of any of claims 1-3 to the subject.

9. A pharmaceutical composition comprising a compound of any of claims 1-3 and a pharmaceutically acceptable excipient.

10. The method according to claims 1-9, wherein a dose of from about 0.0003 to about 30 mg/kg of body weight is administered.

11. A method of diagnosing butyrylcholinesterase activity in a patient which comprises administering to said patient a therapeutically effective amount of a compound selected from the group consisting of Formula (I), Formula (II) or Formula (III): 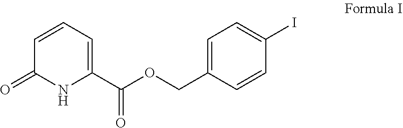 [SMILES: O=C(OCc1ccc(I)cc1)c1cccc(=O)[nH]1]
or a pharmaceutically acceptable salt thereof; 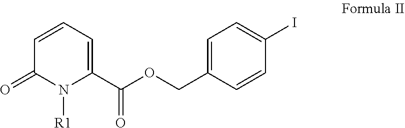
In which R1 is methyl, or a pharmaceutically acceptable salt thereof;

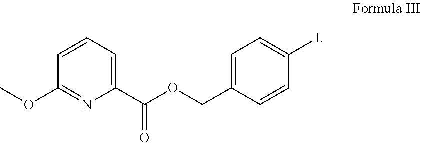 [SMILES: COc1cccc(C(=O)OCc2ccc(I)cc2)n1]

12. A method for diagnosing an amyloid disease in a subject comprising administering to a subject in need thereof a therapeutically effective amount of a compound selected from the group consisting of Formula (I), Formula (II) or Formula (III): 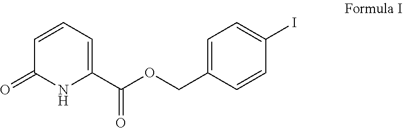 [SMILES: O=C(OCc1ccc(I)cc1)c1cccc(=O)[nH]1]
or a pharmaceutically acceptable salt thereof; 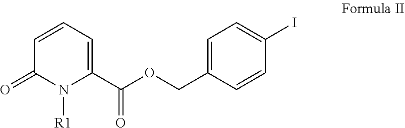
n which R1 is methyl, or a pharmaceutically acceptable salt thereof;

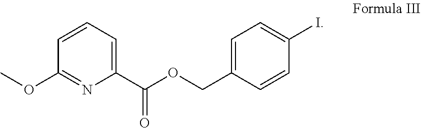 [SMILES: COc1cccc(C(=O)OCc2ccc(I)cc2)n1]

13. The method according to claim 12, wherein said amyloid disease is Alzheimer's disease.

14. The method according to claim 12, wherein said amyloid disease is Parkinson's disease.

15. The method according to claim 12, wherein said amyloid disease is Huntington's disease.

16. The method according to claim 11, wherein a dose of from about 0.0003 to about 30 mg/kg of body weight is administered.

17. The method according to claim 12, wherein a dose of from about 0.0003 to about 30 mg/kg of body weight is administered.
